(19) United States
(12) Patent Application Publication
(10) Pub. No.: US 2022/0087970 A1
(43) Pub. Date: Mar. 24, 2022

(54) PREVENTION OF ANESTHETIC-INDUCED NEUROCOGNITIVE DYSFUNCTION

(71) Applicant: The Johns Hopkins University, Baltimore, MD (US)

(72) Inventors: Roger A. Johns, Baltimore, MD (US); Michele Schaefer, Baltimore, MD (US)

(21) Appl. No.: 17/421,497

(22) PCT Filed: Jan. 18, 2020
(86) PCT No.: PCT/US2020/014233

§ 371 Date: Jul. 8, 2021

Related U.S. Application Data
(60) Provisional application No. 62/794,269, filed on Jan. 18, 2019.

(51) Int. Cl. A61K 31/295; A61K 31/04; A61K 33/00; A61K 31/5377; A61K 31/34; A61K 31/165; A61K 31/506; A61K 31/197; A61K 38/10; A61K 31/4155; A61K 31/397; A61K 31/555; A61K 31/519; A61K 31/4985; A61K 31/53; A61K 31/502; A61K 45/06; A61P 25/28

(52) U.S. Cl. CPC A61K 31/295; A61P 25/28; A61K 33/00; A61K 31/5377; A61K 31/34; A61K 31/165; A61K 31/506; A61K 31/197; A61K 38/10; A61K 31/4155; A61K 31/397; A61K 31/555; A61K 31/519; A61K 31/4985; A61K 31/53; A61K 31/502; A61K 45/06; A61K 31/04

(57) ABSTRACT

Methods for treating or preventing a cognitive impairment in a subject in need of treatment thereof, the method comprising administering to the subject a therapeutically effective amount of an agent that enhances the NO-cGMP-PKG pathway are disclosed.

### FEDERALLY SPONSORED RESEARCH OR DEVELOPMENT

This invention was made with government support under GM110674 awarded by the National Institutes of Health. The government has certain rights in the invention.

### BACKGROUND

Certain general anesthetics have been shown to have adverse effects on neuronal development, including spinogenesis, i.e., the development of dendritic spines in neurons, and synaptogenesis, i.e., the formation of synapses between neurons in the nervous system, which affect neural function and cognitive behavior. Further, there is accumulating evidence that early exposure to anesthetic agents may interfere with brain development and ultimately lead to permanent cognitive deficits. As a result, the United States Food and Drug Administration has identified pediatric anesthetic neurotoxicity (PAN) as a potentially important health issue. The molecular mechanisms that underlie PAN and other anesthetic-induced cognitive impairments are still poorly understood.

### SUMMARY

The presently disclosed subject matter provides a method for treating or preventing a cognitive impairment in a subject, the method comprising administering to the subject an agent that enhances the NO-cGMP-PKG pathway. In certain embodiments, the agent that enhances the NO-cGMP-PKG pathway includes an NO donor, a guanylate cyclase activator, and a type 5 phosphodiesterase (PDE5) inhibitor.

In particular aspects, the presently disclosed subject matter provides a method for treating or preventing a cognitive impairment in a subject in need of treatment thereof, the method comprising administering to the subject a therapeutically effective amount of at least one nitric oxide (NO) donor.

In certain aspects, the cognitive impairment is associated with one or more surgical procedures. In particular aspects, the cognitive impairment is anesthetic induced. In more particular aspects, the anesthetic is a general anesthetic. In certain aspects, the general anesthetic is selected from the group consisting of an inhalational anesthetic, an injectable anesthetic, and combinations thereof.

In some aspects, the cognitive impairment is selected from the group consisting of an impaired memory, an impaired object recognition memory, a learning disability, and an attention deficit/hyperactivity disorder. In other aspects, the method further comprises one or more conditions or disorders selected from the group consisting of anxiety and emotional reactivity.

In particular aspects, the cognitive impairment is associated with a change or impairment in dendritic spine morphology or development; plasticity; neural plasticity; long-term potentiation (LTP), neuronal apoptosis, and combinations thereof. In yet more particular aspects, the cognitive impairment is associated with a disruption of PSD-95 discs large homolog, and zona occludens-1 (PDZ)2 domain-mediated protein-protein interactions and/or a N-methyl-D aspartate (NMDA) receptor/PSD-95 PDZ2/neuronal nitric oxide synthase (nNOS) signaling pathway.

In certain aspects, the at least one NO donor is selected from the group consisting of sodium nitroprusside (SNP), nitroglycerin (NTG), an organic nitrate, a sydnonimine, a diazeniumdiolate, an S-nitrosothiol, such as, S-nitroso-N-acetyl-D, L-penicillamine (SNAP), and nitric oxide (NO). In particular aspects, the at least one NO donor is a sydnonimine. In yet more particular aspects, the sydnonimine is molsidomine or isosorbide. In other aspects, the method further comprises a pharmaceutical formulation comprising molsidomine, isosorbide, or other NO donor.

In some aspects, the at least one NO donor is administered in combination with one or more anesthetics. In particular aspects, the at least one NO donor is administered before, after, or concurrently with one or more anesthetics.

In certain aspects, the subject is selected from the group consisting of a neonate, an infant, a one- to three-year old child, an unborn fetus, and a patient who is pregnant.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIG. 1 (prior art) is a diagram illustrating dissociation of NMDAR-PSD95/93*-nNOS interaction by ISO or PDZ2WT (active) peptide. *For simplicity, not all PSD-95 family members are represented. Before Exposure to Isoflurane (ISO): The NMDA receptor is linked to downstream molecules such as nNOS through PSD-95: through its first and second PDZ domain, PSD-95 forms a ternary complex by binding to both the tSXV motif of NMDAR NR2 subunit and to the PDZ domain in nNOS. Sattler et al., 1999. Disrupting NMDAR-PSD-95/93-nNOS complexes can reduce the efficiency by which calcium ions activate the signaling molecule nNOS. After Exposure to ISO: This disruption is achieved by exposure to inhalational anesthetics, Fang et al., 2003; Tao et al., 2015, or the intracellular introduction of PDZ2WT peptide Tao et al., 2008; this is expected to bind to NMDAR NR2. Aarts et al., 2002. $ Inhalational anesthetics (and presumably PDZ2WT peptide) also can inhibit interactions between PSD-95 PDZ2 domain and Shaker type potassium channel Kv1.4, as well as other excitatory receptor channels related to anesthesia and other proteins not shown here for simplicity. Tao et al., 2015;

FIG. 2A and FIG. 2B are western blot assays that did not show any significant differences in apoptosis in our exposure paradigm. Post-natal day (PND) 7 mice were exposed to isoflurane for 4-hours and their brains harvested 2-hours after cessation of exposure. FIG. 2A, Western blot analysis for total caspase-3 revealed the presence of procaspase 3, but did not reveal any detectable cleaved caspase-3 in mice exposed to CON (O₂), ISO (1.5% ISO in 100% 02), PDZ2MUT (8 mg/kg inactive peptide), PDZ2WT (8 mg/kg active peptide). Negative and positive Jurkat control proteins were run to demonstrate sensitivity of the caspase-3 antibody. FIG. 2B, Western blot analysis for PARP did not reveal significant differences between CON (O₂) and ISO (1.5% ISO in 100% 02). Left, representative blot. Right, Densitometry data are plotted as median, interquartile range. CON (n=6) vs ISO (n=6), p=0.818. Data were analyzed using Mann Whitney test. Both males and females were studied;

FIG. 3A, FIG. 3B, and FIG. 3C show neonatal exposure to ISO or PDZ2 wild-type (WTO peptide alters hippocampal dendritic spine morphology and development. FIG. 3A, dorsal hippocampal region of a Golgi preparation illustrating dendrites from the superior blade of the dentate gyrus (DG) subregion of interest (white box); scale bar represents 250 μm. FIG. 3B, schematics showing dendrite branches and spine types sampled and a representative dendritic segment with spines; scale bar represents 2.5 μm. Spines were assessed on dendritic segments distal to the first and second branch points. FIG. 3C, Distribution of dendritic spines according to morphological type in DG among exposed groups assessed at PND21. WT and PSD-93 knock-out (KO) PND7 mice were exposed to O₂ (control, 100% 02) or ISO (1.5% ISO in 100% O₂); WT PND7 mice were also injected with PDZ2 mutant (MUT) (8 mg/kg inactive peptide) or PDZ2WT (8 mg/kg active peptide). (WT control (CON)=8, WT ISO=4, PSD93KO CON=4, PSD93KO ISO=5, PDZ2MUT=4, PDZ2WT=4). Data from individual animals are plotted and color coded by gender (red=female and blue=male). Data are plotted as median number of spines per micron with interquartile range). Data were analyzed with Kruskal-Wallis followed by Dunn's multiple comparison correction and Mann-Whitney tests. A *p<0.05 was considered significant;

FIG. 4A, FIG. 4B, and FIG. 4C show neonatal exposure to ISO or PDZ2WT peptide did not have an acute impact on the number of hippocampal postsynaptic densities (PSDs). FIG. 4A, dorsal hippocampal region of a semi-thin section illustrating DG subregion of interest (white box). FIG. 4B, representative ultrastructure images from PND21 mice exposed at PND7 to 02 (WT CON, 100% 02), WT ISO (1.5% ISO in 100% O₂), PDZ2MUT peptide (8 mg/kg inactive peptide), or PDZ2WT peptide (8 mg/kg active peptide); scale bar represents 500 nm. Asterisks showing some PSDs (not all are labeled). FIG. 4C, plots showing the median with interquartile range number of PSD's. WT CON (n=6) vs WT ISO (n=5), p=0.829; PDZ2MUT (n=4) vs PDZ2WT (n=4), p=0.742. Data from individual animals are plotted and color coded by gender (red=female and blue=male) Data were analyzed with Mann-Whitney;

FIG. 5A and FIG. 5B show neonatal exposure to ISO or PDZ2WT peptide impairs long-term potentiation (LTP) in hippocampal CA1 at PND21. FIG. 5A, high frequency stimulation (HFS) induced robust LTP in WT CON (top; WT CON, 100% 02) and inactive PDZ2MUT (bottom) treated groups in hippocampal Schafer collateral to CA1 pathway. ISO (top) and PDZ2WT (bottom) exposure as well as PSD93 deficiency (middle) impaired the expression of LTP. Example traces are shown in upper left quadrants of fEPSP plots. WT CON and WT ISO (top), PSD93KO CON and PSD93KO ISO (middle), and PDZ2MUT and PDZ2WT (bottom) treated groups at baseline before HFS (solid line trace), and the average of 55-60 min after HFS (dashed line trace). FIG. 59, the median of normalized fEPSP 55-60 min after HFS showed significant differences between WT CON (n=7) vs WT ISO (n=5), p=0.049 and PDZ2MUT (n=4) vs PDZ2WT (n=4), p=0.028 treated groups. Significant differences were not observed between WT CON vs PSD93KO CON (n=5), p=0.056 but were observed between WT CON vs PSD93KO ISO (n=8), p=0.025 groups. Data from individual animals are plotted and color coded by gender (red=female and blue=male). Data were analyzed with Kruskal-Wallis followed by post-hoc Dunn's test and Mann Whitney tests. Values were considered significant at *p<0.05 or less. Data were plotted as median and interquartile range. Scale bar: 10 ms, 0.25 mV;

FIG. 6A and FIG. 6B show neonatal exposure to ISO or PDZ2WT peptide causes a subtle but significant decrease in acute recognition memory. FIG. 6A, plots showing percent of time animals spent investigating novel or known objects among experimental groups (WT Naïve, n=11, p<0.0001; WTCON, n=14, p<0.0001; WT ISO, n=18, p=0.005; PSD93KO CON, n=10, p=0.001; PDZ2MUT, n=14, p<0.0001; PDZ2WT, n=16, p=0.001). The double hit animals were unable to significantly discriminate between novel and known objects (PSD93KO ISO, n=10, p=0.098; PDZ2WT+ISO, n=8, p=0.227). Data were plotted as mean and SD. Data were analyzed with two-tailed t-tests known vs. novel. FIG. 6B, plots showing discrimination index as percent of time animals spent investigating novel object over the total time investigating novel and known objects multiplied by 100. ISO-exposed WT animals have a subtle but significant decrement in recognition memory as compared to controls (WT NAÏVE vs WT ISO, p=0.022; WT CON vs WT ISO, p=0.043; WT NAÏVE vs WTCON, p>0.999). PSD93 deficiency did not have a significant effect on recognition memory (WT NAÏVE vs PSD93KO CON, p>0.999; WT CON vs PSD93KOCON, p>0.999). ISO-exposed PSD93 KO animals were not significantly different from PSD93 KO controls (PSD93KO CON vs PSD93KO ISO, p=0.176). ISO-exposed PSD93KO animals differed from WT controls (WT NAÏVE vs PSD93KO ISO, p=0.011; WT CON vs PSD93KO ISO, p=0.022). Active peptide exposed animals have a subtle but significant decrement in recognition memory as compared to inactive peptide controls (PDZ2MUT vs PDZ2WT, p=0.038; PDZ2MUT vs PDZ2WT+ISO, p<0.001) ISO exposure did not further significantly impair peptide exposed animals (PDZ2WT vs PDZ2WT+ISO, p=0.385). Data from individual animals are plotted and color coded by gender (red=female and blue=male). Data were analyzed with Kruskal-Wallis followed by post-hoc Dunn's test. Data were plotted as median and interquartile range. *p<0.05, **p<0.01, ***p<0.001, and ****p<0.0001;

FIG. 7A and FIG. 7B show treatment with NO donor, e.g., molsidomine, prevents the negative effect of ISO and PDZ2WT peptide on hippocampal LTP. In this experiment, the NO donor was added immediately following cessation of anesthesia or control (O₂) exposure, i.e., the NO donor was injected 4 hours after the onset of anesthesia. FIG. 7A, robust LTP was induced by HFS in all groups. In upper left quadrants example traces of WT CON+NO and WT ISO+NO (top), PDZ2MUT+NO and PDZ2WT+NO (bottom) treated groups before HFS (solid line trace) and 55-60 min after HFS (dashed line trace) are shown. FIG. 7B, the median of normalized fEPSP 55-60 min after HFS no longer shows a significant difference between WT CON and WT ISO when NO donor is added (WT CON+NO (n=5) vs ISO+NO (n=8), p=0.284) or between PDZ2MUT and PDZ2WT (PDZ2MUT+NO (n=5) vs PDZ2WT+NO (n=6), p=0.662). Data were analyzed with Mann-Whitney tests. Data from individual animals are plotted and color coded by gender (red=female and blue=male). Data were plotted as median and interquartile range. Values were considered significant at *p<0.05 or less. Scale bar: 10 ms, 0.25 mV;

FIG. 8 shows treatment with NO donor, e.g., molsidomine, prevents ISO or PDZ2WT peptide induced impairment in acute recognition memory. Plots showing discrimination index as percent of time animals spent investigating novel object over the total time investigating novel and known objects multiplied by 100. No significant difference between WT CON and WT ISO was observed when NO donor is added (WT CON+NO (n=6) vs ISO+NO (n=7), p=0.073) or between PDZ2MUT and PDZ2WT (PDZ2MUT+NO (n=8) vs PDZ2WT+NO (n=7), p=0.778). Data from individual animals are plotted and color coded by gender (red=female and blue=male). Data were plotted as median and interquartile range. Data were analyzed with Mann-Whitney tests. Values were considered significant at *p<0.05 or less;

FIG. 9 show the neonatal exposure to isoflurane or PDZ2WT peptide decreases mature hippocampal mushroom spine density in adult mice. Density of mushroom spines among exposed groups assessed at PND49. Mice in cohort 1 were exposed to O₂ (control, 100% 02) or ISO (1,5% ISO in 02) and cohort 2 were injected with PDZ2MUT (8 mg/kg inactive peptide) or PDZ2WT (8 mg/kg active peptide). Data are plotted as box whisker plots showing density (# spines per micron) median, IQ range, minimum and maximum. Left panel, both genders (CON=16, ISO=11, PDZ2MUT=14, PDZ2WT=16). Middle panel, females (CON=6, ISO=5, PDZ2MUT=4, PDZ2WT=8). Right panel, males (CON=10, ISO=6, PDZ2MUT=10, PDZ2WT=8). Data were analyzed with two-tailed Mann Whitney tests. *p<0.05, **p<0.01, ***p<0.001, ****p<0.0001;

FIG. 10 shows the neonatal exposure to isoflurane or PDZ2WT peptide impairs recognition memory at PND42. Plots showing percent of time animals spent investigating novel or known objects among experimental groups. On PND7, mice in cohort 1 were exposed to O₂ (control, 100% 02) or isoflurane (1.5% ISO in 02) and cohort 2 were injected with PDZ2MUT (8 mg/kg inactive peptide) or PDZ2WT (8 mg/kg active peptide). Data are plotted as median and IQ range. Left panel, both genders (Naïve, n=13, p=0.0002; CON=25, p=0.0002; ISO=20, p=0.0574; PDZ2MUT, n=20, p=0.0010; PDZ2WT, n=19, p=0.3799). Middle panel, females (Naïve, n=5, p=0.0159; CON, n===10, p=0.0147; ISO, n=10, p=0.6842; PDZ2MUT, n=11, p=0.0158; PDZ2WT, n=10, p=0.7959). Right panel, males (Naïve, n=8, p=0.0104; CON, n=15, p=0.0048; ISO, n=10, p=0.1273; PDZ2MUT, n=9, p=0.0061; PDZ2WT, n=9, p=0.5613). Data were analyzed with two-tailed. Mann Whitney tests, known vs. novel. *p<0.05, **p<0.01, ***p<0.001;

FIG. 11 shows the neonatal exposure to isoflurane or PDZ2WT peptide alters simple Y-maze memory at PND42. Plots showing percent of time animals spent investigating novel or known arms among experimental groups. On PND7, mice in cohort 1 were exposed to O₂ (control, 100% 02) or isoflurane (1.5% ISO in 02) and cohort 2 were injected with PDZ2MUT (8 mg/kg inactive peptide) or PDZ2WT (8 mg/kg active peptide). Data are plotted as median and IQ range. Left panel, both genders (CON=26, p<0.0001; ISO=15, p=0.0145, PDZ2MUT, n=20, p<0.0001; PDZ2WT, n=20, p=0.0035). Middle panel, females (CON, n=15, p=0.0115; ISO, n=7, p=0.3829; PDZ2MUT, n=8, p=0.0011; PDZ2WT, n=9, p=0.1359). Right panel, males (CON, n=11, p=0.0019; ISO, n=8, p=0.0379; PDZ2MUT, n=12, p=0.0001; PDZ2WT, n===11, p=0.0122). Data were analyzed with two-tailed Mann Whitney tests, known vs. novel. *p<0.05**p<0.01, ***p<0.001, ****P<0.0001;

FIG. 12 shows the neonatal exposure to isoflurane or PDZ2WT peptide impairs fear memory at PND56. On PND7, mice in cohort 1 were exposed to O₂ (control, 100% O₂) or isoflurane (1.5% ISO in 02) and cohort 2 were injected with PDZ2MUT (8 mg/kg inactive peptide) or PDZ2WT (8 mg/kg active peptide). Data are plotted as box whisker plots showing % freezing duration median, IQ range, minimum and maximum. Left panel, both genders (CON=23, ISO=30, PDZ2MUT=23, PDZ2WT=24). Middle panel, females (CON=12, ISO=15, PDZ2MUT=10, PDZ2WT=12). Right panel, males (CON=11, ISO=15, PDZ2MUT=13, PDZ2WT=12). Data were analyzed with two-tailed Mann Whitney tests *P<0.05, **P<0.01, ***P<0.001, ****P<0.0001;

FIG. 13A and FIG. 13B show the neonatal exposure to isoflurane or PDZ2WT peptide does not result in long lasting impairment in long-term potentiation (LTP) in hippocampal CA1 at PND49. On PND7, mice in cohort 1 were exposed to O₂ (control, 100% 02) or isoflurane (1.5% ISO in 02) and cohort 2 were injected with PDZ2MUT (8 mg/kg inactive peptide) or PDZ2WT (8 mg/kg active peptide). FIG. 13A, high frequency stimulation (HFS) induced robust LTP in all groups. Top panel, CON=6 and ISO=5. Bottom panel, PDZ2MUT=4 and PDZ2WT=4. Example traces are shown in upper left quadrants of fEPSP plots. CON and ISO (top) and PDZ2MUT and PDZ2WT (bottom) treated groups at baseline before HFS (solid line trace) and the average of 55-60 min after HFS (dashed line trace). FIG. 13B, the median of normalized fEPSP 55-60 min after HFS showed no significant differences between CON (n=6) vs ISO (n=5), p=0.4286 and PDZ2MUT (n=4) vs PDZ2WT (n=4), p=0.4857 treated groups. Data from individual animals are plotted and coded by gender (gray=female and black=male). Data were analyzed with two-tailed Mann Whitney tests. Values were considered significant at *p<0.05 or less. Data (normalized fEPSP 55-60 min after HFS) are plotted as box whisker plots showing median, IQ range, minimum and maximum. Scale bar: 10 ms, 0.25 mV;

FIG. 14 shows the treatment with NO donor prevents the decrease in mushroom spine density in PND49 adult mice induced by neonatal exposure to isoflurane or PDZ2WT peptide. Density of mushroom spines among exposed groups assessed at PND49. On PND7, mice in cohort 1 were exposed to O₂ (control, 100% 02) or isoflurane (1.5% ISO in 02) and cohort 2 were injected with PDZ2MUT (8 mg/kg inactive peptide) or PDZ2WT (8 mg/kg active peptide). At the end of the exposure period (four hours after onset of exposure) all animals received 4 mg/kg of NO donor molsidomine (ip). Data are plotted as box whisker plots showing density (# spines per micron) median, IQ range, minimum and maximum. Left panel, both genders (CON, n=12 vs ISO, n=13), p=0.0678; (PDZ2MUT, n=12 vs PDZ2WT, n=12), p=0.6297. Middle panel, females (CON, n=6 vs ISO, n=6), p=0.3095; (PDZ2MUT, n=6 vs PDZ2WT, n=6), p=0.4848. Right panel, males (CON 6, ISO=7), p=0.1807; (PDZ2MUT, n=6 vs PDZ2WT, n=6), p>0.9999. Data were analyzed with two-tailed Mann Whitney tests. *p<0.05, **p<0.01, ***p<0.001, ****p<0.0001;

FIG. 15A and FIG. 15B show that isoflurane exposure and disrupting PDZ domain interactions impairs dendritic spine development in vivo as seen by a loss of mushroom ‘mature’ spines at 7-weeks of age and can be prevented with NO donor. FIG. 15A, density of mushroom spines in DG among exposed groups assessed at 7 weeks of age. (N=10-14). FIG. 15B, introduction of NO donor prevents loss of mushroom spines in experimental animals (ISO and PDZ2WT) making them comparable in density to controls (CON and PDZ2MUT). (N=4-7). A *p<0.05 was considered significant;

FIG. 16 shows that disrupting PDZ domain interactions attenuates phosphorylation of VASP. One day after PND7 mice were exposed to PDZ2WT or PDZ2MUT peptides (8 mg/kg), we harvested hippocampal tissues for Western blotting to assess the phosphorylation status of VASP. PDZ2WT peptide exposure resulted in a significant decrease in the amount of pVASP as compared to inactive PDZ2MUT peptide control. (N=3). A *p<0.05 was considered significant;

FIG. 17 shows that activation of NO-cGMP-PKG pathway enhances phosphorylation of ERK. PND7 mice were exposed to isoflurane (1.5% ISO in 02, 4 hr), control gas (100% O₂, 4 hr), PDE inhibitor (BAY 60-7550, 3 mg/kg, 30 min), or NO donor (molsidomine, 4 mg/kg, 30 min), we harvested hippocampal tissues for Western blotting to assess the phosphorylation status of ERK. PDE inhibitor and NO donor resulted in significant increases in the amount of pERK;

FIG. 18A, FIG. 18B, FIG. 18C, and FIG. 18D show that isoflurane exposure results in overgrowth of dendritic arbors. Kang et al., 2017. Representative confocal images (FIG. 18A) and tracings (FIG. 18B) of individual control and isoflurane exposed GFP+ neurons at PND30 exhibiting overgrowth in the isoflurane group relative to control conditions (scale bar: 10 μm). Summaries of total dendritic length (FIG. 18C) and Sholl analysis of dendritic complexity (FIG. 18D) show marked overgrowth of dendritic arbors (N≥5). Data indicates early exposure to isoflurane alters hippocampal dendritogenesis and this enhanced arborization is still present at PND30.pared to control. (N=3). A *p<0.05 was considered significant. These results indicate that activation of NO-cGMP-PKG leads to increases in ERK phosphorylation which suggests that inhibition of the pathway by isoflurane would attenuate pERK;

FIG. 19 shows that isoflurane exposure extends presence of NR2B and SAP102 in the synapse. One day after PND7 mice were exposed to isoflurane (1.5% ISO in O₂, 4 h), we harvested hippocampal tissues for Western blotting to assess the expression of receptors and MAGUKs. Isoflurane exposure resulted in a significant increase in the amount of NR2B and SAP102 and decrease in NR1 in synaptic fractions. (N=9). A *p<0.05 was considered significant. These results suggest that the normal developmental decline of NR2B and SAP102 may be impeded resulting in their prolonged presence at the synapse. Data shown as % actin;

FIG. 20A and FIG. 20B show that isoflurane exposure impairs dendritic spine development in vitro as seen by a loss of long thin ‘immature’ spines and filopodia at DIV7 and can be prevented with NO donor and prevention blocked with sGC inhibitor. Primary neuron cultures were exposed to control gas (25% O₂, 5% CO₂, bal N₂), isoflurane (1.5% ISO in 25% O₂, 5% CO₂, bal N₂), DETA NONOate NO donor 150 μM and sGC inhibitor ODQ 100 μM for 4 hrs. FIG. 20A, Representative images showing the loss of long thin and filopodia spines after isoflurane exposure. Treatment of cultures with NO donor prevented loss of immature spines. Addition of sGC inhibitor blocked the prevention with NO donor. MAP2 dendrite marker, green. Drebrin A/E immature spine/filopodia marker, red. Scale bar is 5 μm. FIG. 20B, Density of long thin spines and filopodia at DIV7 among exposed groups analyzed using Imaris 93.1;

FIG. 21A and FIG. 21B show that disrupting PDZ domain interactions impairs dendritic spine development in vitro as seen by a loss of long thin ‘immature’ spines and filopodia at DIV7 and can be modulated with NO donor and sGC inhibitor. Neuron cultures were exposed to inactive PDZ2MUT peptide (1 μM), active PDZ2WT peptide (1 μM), DETA NONOate NO donor 150 μM, and sGC inhibitor ODQ 100 μM for 4 hrs. FIG. 21A shows representative images showing the loss of long thin and filopodia spines after PDZ2WT exposure. Treatment with NO donor prevented loss of immature spines. Addition of sGC inhibitor blocked the prevention with NO donor. MAP2, green. Drebrin, red. Scale bar is 5 μm. FIG. 21B, show the density of long thin spines and filopodia among exposed groups;

FIG. 22A and FIG. 22B demonstrates that sevoflurane exposure induces a loss of filopodia at DIV7. Neuron cultures were exposed to control gas (25% O₂, 5% CO₂, balance N₂) or sevoflurane (2.1% ISO in 25% O₂, 5% CO₂, balanced N₂) for 4 hrs. FIG. 22A, Representative images showing loss of filopodia after sevoflurane exposure. Scale bar is 5 μm. FIG. 22B, Density of long thin spines and filopodia among exposed groups; and

FIG. 23A and FIG. 23B demonstrate that isoflurane exposure causes loss of synapses at DIV14, which can be prevented with an NO donor and this prevention is attenuated with an sGC inhibitor. Neuron cultures were transfected with GFP-AAV virus at DIV-4 and were exposed to NO donor (DETA) 150 μM and sGC inhibitor (ODQ) 100 μM for 4 hrs right before exposure to gases at DIV7. Cells were returned back to incubator with half media change every second day. At DIV14, cells were fixed and stained with PSD95 and Synaptophysin. FIG. 23A: Representative images show the loss of synapse after isoflurane exposure. Treatment with NO donor prevented loss of synapses. Addition of ODQ blocked the prevention with DETA. Scale bar is 5 μm. FIG. 23B: Synapse were analyzed using puncta analyzer plugin as shown in methods section.

### DETAILED DESCRIPTION

The presently disclosed subject matter now will be described more fully hereinafter with reference to the accompanying Examples and Figures, in which some, but not all embodiments of the presently disclosed subject matter are illustrated. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Examples and Figures. Therefore, it is to be understood that the presently disclosed subject matter is not to be limited to the specific embodiments disclosed and that modifications and other embodiments are intended to be included within the scope of the appended claims.

### I. Prevention of Anesthetic-Induced Neurocognitive Dysfunction

The presently disclosed subject matter provides methods for preventing anesthetic-induced effects on memory and cognition pathways in the central nervous system, including hippocampal long-term potentiation (LTP) and recognition memory, by administering an agent that enhances the NO-cGMP-PKG pathway before, after, or concurrently with an anesthetic. In certain embodiments, the agent that enhances the NO-cGMP-PKG pathway includes an NO donor, a guanylate cyclase activator, and a type 5 phosphodiesterase (PDE5) inhibitor.

More particularly, the presently disclosed subject matter demonstrates that treatment with an agent that enhances the NO-cGMP-PKG pathway, including, but not limited to, an NO donor, a guanylate cyclase activator, and a type 5 phosphodiesterase (PDE5) inhibitor, prevents neonatal anesthetic-induced impairments in synaptic plasticity and memory. Further, anesthetic-induced alterations in dendritic spine morphology and function important to cognition, neural plasticity (i.e., LTP) and impairment of object recognition memory can be prevented by administering an agent that enhances the NO-cGMP-PKG pathway, including, but not limited to, an NO donor, a guanylate cyclase activator, and a type 5 phosphodiesterase (PDE5) inhibitor, to a subject in need of treatment thereof.

Accordingly, in some embodiments, the presently disclosed subject matter provides a method for treating or preventing a cognitive impairment in a subject in need of treatment thereof, the method comprising administering to the subject a therapeutically effective amount of an agent that enhances the NO-cGMP-PKG pathway. In particular embodiments, the agent that enhances the NO-cGMP-PKG pathway is selected from the group consisting of an NO donor, a guanylate cyclase activator, and a type 5 phosphodiesterase (PDE5) inhibitor.

In some embodiments, the cognitive impairment is associated with one or more surgical procedures. In particular embodiments, the cognitive impairment is anesthetic induced.

In certain embodiments, the anesthetic is a general anesthetic or a regional anesthetic. In particular embodiments, the general anesthetic is selected from the group consisting of an inhalational anesthetic, an injectable anesthetic, and combinations thereof. In more particular embodiments, the inhalational anesthetic is selected from the group consisting of isoflurane ((RS)-2-chloro-2-(difluoromethoxy)-1,1,1-trifluoroethane), halothane (2-bromo-2-chloro-1,1,1-trifluoroethane), sevoflurane (1,1,1,3,3,3-hexafluoro-2-(fluoromethoxy)propane), desflurane (1,2,2,2-tetrafluoroethyl difluoromethyl ether), enflurane (2-chloro-1,1,2,-trifluoroethyl-difluoromethyl ether), methoxyflurane (2,2-dichloro-1,1-difluoroethyl methyl ether), nitrous oxide, xenon, and combinations thereof. In other embodiments, the injectable anesthetic is selected from the group consisting of propofol (2,6-diisopropylphenol), etomidate (ethyl 3-[(1R)-1-phenylethyl]imidazole-5-carboxylate), ketamine ((RS)-2-(2-chlorophenyl)-2-(methylamino)cyclohexanone), a barbiturate, such as methohexital and thiopentone/thiopental, a benzodiazepine, such as midazolam, and combinations thereof. In certain embodiments, the regional anesthetic is selected from the group consisting of a nerve block, a spinal anesthetic, an epidural anesthetic, and a caudal anesthetic. The presently disclosed methods are suitable for use with other anesthetics known in the art, as well.

In some embodiments of the presently disclosed methods, the cognitive impairment is selected from the group consisting of an impaired memory, an impaired object recognition memory, a learning disability, and an attention deficit/hyperactivity disorder (ADHD). As used herein, the term “memory” can include working memory, short-term memory, and/or long-term memory. As used herein, the term “object recognition memory” refers to the ability to judge a previously encountered object as familiar. Learning disabilities include, but are not limited to, central auditory processing disorder, dyscalculia, dysgraphia, dyslexia, language processing disorder, a non-verbal learning disability, and a visual perception/visual motor deficit disorder.

In some embodiments, the presently disclosed methods further comprise treating or preventing one or more conditions or disorders selected from the group consisting of anxiety and emotional reactivity. As used herein, the term “emotional reactivity” refers to involuntary and usually overly intense reaction to an external emotional stimulus.

In particular embodiments, the cognitive impairment is associated with a change or impairment in dendritic spine morphology or development, including spinogenesis; synaptic plasticity; neural plasticity; long-term potentiation (LTP), neuronal apoptosis, and combinations thereof. Such changes or impairment can occur in one or more neuronal regions including, but not limited to, the hippocampus, the cortex, and the amygdala.

As used herein, the term “synaptic plasticity” refers to the ability of synapses to strengthen or weaken over time, in response to increases or decreases in their activity. As used herein, the term “long-term potentiation (LTP)” refers to a persistent strengthening of synapses based on recent patterns of activity, such as high-frequency stimulation, especially in the hippocampus, a small region of the brain that is primarily associated with memory and spatial navigation. As used herein, the term “neural plasticity” can be defined as the ability of the central nervous system (CNS), e.g., neurons, to adapt in form and function in response to changes in their environment. As used herein, the term “apoptosis” refers to a form of programmed cell death.

As provided in more detailed herein below, for example, in Example 1, in certain embodiments, the cognitive impairment is associated with a disruption of PSD-95 discs large homolog, and zona occludens-1 (PDZ)2 domain-mediated protein-protein interactions and/or a N-methyl-D aspartate (NMDA) receptor/PSD-95 PDZ2/neuronal nitric oxide synthase (nNOS) signaling pathway.

As used herein, the term “nitric oxide donor” or “NO donor” and the like refers to any substance that is converted into, degraded or metabolized into, or donates, releases, and/or directly or indirectly transfers or otherwise provides a source of a nitrogen monoxide species (⋅N═O) in vivo or under physiological conditions and/or stimulates the endogenous production of nitric oxide in vivo and/or elevates endogenous levels of nitric oxide in vivo and/or are oxidized to produce nitric oxide and/or are substrates for nitric oxide synthase (nNOS). The term NO donor encompasses any compound that generates or releases NO through biotransformation, any compound that generates NO spontaneously, and any compound that generates NO in any manner when administered to a subject. In some embodiments, the NO donor is nitric oxide.

Nitric oxide donors include several classes of compounds having differing structural features. Representative NO donors and classes of NO donors include, but are not limited to:

Sodium nitroprusside (SNP);

Organic nitrates including, but not limited to, nitroglycerin (or glyceryl trinitrate (GTN)), clonitrate, isosorbide-5-mononitrate (ISMN), isosorbide dinitrate (ISDN), [N-[2-(nitroxyethyl)]-3-pyridinecarboxamide (nicorandil), pentaerythritol tetranitrate (PETN), Pentaerythritol trinitrate, propatylnitrate, and erythrityl tetranitrate (ETN);

mannitol hexanitrate; NORS (sodium nitrite citric acid); naproxcinod;

Sydnonimines including, but not limited to, molsidomine (N-ethoxycarbonyl-3-morpholinosydnonimine), SIN-1 (3-morpholinosydnonimine or linsidomine) CAS 936 (3-(cis-2,6-dimethylpiperidino)-N-(4-methoxybenzoyl)-sydnonimine, pirsidomine), C87-3754 (3-(cis-2,6-dimethylpiperidino)sydnonimine, C4144 (3-(3,3-dimethyl-1,4-thiazane-4-yl)sydnonimine hydrochloride), C89-4095 (3-(3,3-dimethyl-1,1-dioxo-1,4-thiazane-4-yl)sydnonimine hydrochloride, CAS 754, feprosidnine, and the like;

S-nitrosothiols including, but not limited to, S-nitroso-N-acetylpenicillamine (SNAP), S-nitroso-glutathione, and S,S-dinitrosodithiol (SSDD);

S-nitrothiols; nitrites; nitrates;

Diazeniumdiolates, such as 2-hydroxy-2-nitrosohydrazines (NONOates) including, but not limited to, (Z)-1-(N-methyl-N-(6-(N-methyl-ammoniohexyl)amino))diazen-1-ium-1,2-diolate (“MAHMA/NO”); (Z)-1-(N-(3-ammoniopropyl)-N-(n-propyl)amino)diazen-1-ium-1,2-diolate (“PAPA/NO”); (Z)-1-(N-(3-aminopropyl)-N-(4-(3-aminopropylammonio)butyl)-amino) diazen-1-ium-1,2-diolate (spermine NONOate or “SPER/NO”); di sodium 1-[(2-carboxylato)pyrrolidin-1-yl]diazen-1-ium-1,2-diolate methanol (PROLI-NONOate), (O₂-(2,4-Dinitrophenyl) 1-[(4-ethoxycarbonyl)piperazin-1-yl]diazen-1-ium-1,2-diolate (JS-K), and sodium(Z)-1-(N,N-diethylamino)diazenium-1,2-diolate (diethylamine NONOate or “DEA/NO”) and derivatives thereof. Representative NONOates also are described in U.S. Pat. Nos. 6,232,336, 5,910,316 and 5,650,447, the disclosures of which are incorporated herein by reference in their entirety;

(E)-alkyl-2-((E)-hydroxyimino)-5-nitro-3-hexeneamide (FK-409); (E)-allyl-2-((E)-hydroxyimino)-5-nitro-3-hexeneamines;

N-((2Z, 3E)-4-ethyl-2-(hydroxyimino)-6-methyl-5-nitro-3-heptenyl)-3-pyridinecarboxamide (FR 146801);

N-nitrosoamines; N-hydroxyl nitrosamines; nitrosimines; diazetine dioxides; oxatriazole 5-imines;

oximes including, but not limited to, NOR-1, NOR-3, NOR-4, and the like;

hydroxylamines: N-hydroxyguanidines: Hydroxyureas;

Furoxans (1,2,5-oxadiazole 2-oxide) including, but not limited to, CAS 1609, C93-4759, C92-4678, S35b, CHF 2206, CHF 2363, and the like;

[3-(1H-Imidazol-4-yl)propyl]guanidines-containing furoxan moieties; Benzofuroxans: NO-donor phenols;
pseudojujubogenin glycosides, such as dammarane-type triterpenoid saponins (e.g., bacopasaponins), as well as their derivatives or analogs;

amino acid derivatives, such as N-hydroxy-L-arginine (NOHA), N₆-(1-iminoethyl)lysine) (L-NIL), L-N₅-(1-iminoethyl)omithine (LN-NIO), N-methyl-L-arginine (L-NMMA), and S-nitroso amino acids, such as S-nitroso-N-acetylcysteine, S-nitroso-captopril, S-nitroso-N-acetylpenicillamine, S-nitroso-homocysteine, S-nitroso-cysteine, S-nitroso-glutathione (SNOG), S-nitroso-cysteinyl-glycine, SPM 5185 (N-nitratopivaloyl-S—(N′-acetylalanyl)-cysteine ethyl ester), SPM 3672 (N-(3-nitrato-pivaloyl)-1-cysteineethylester), and the like;

metabolic precursors of NO, including, but not limited to, L-arginine and L-citrulline; and

substrates for the endogenous enzymes which synthesize nitric oxide.

In particular embodiments, the nitric oxide donor is molsidomine:

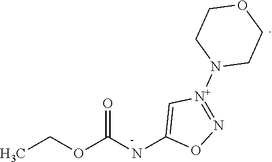 [SMILES: CCOC(=O)Nc1c[n+](N2CCOCC2)no1]

Molsidomine is a vasodilator belonging to the chemical class of sydnonimines. It is metabolized in the liver to SIN-1 (3-morpholinosydnonimine, linsidomine), which spontaneously hydrolyses to the nitroso metabolite, SIN-1A (N-nitroso-N-morpholino-amino-acetonitrile), the active metabolite in blood that releases NO:

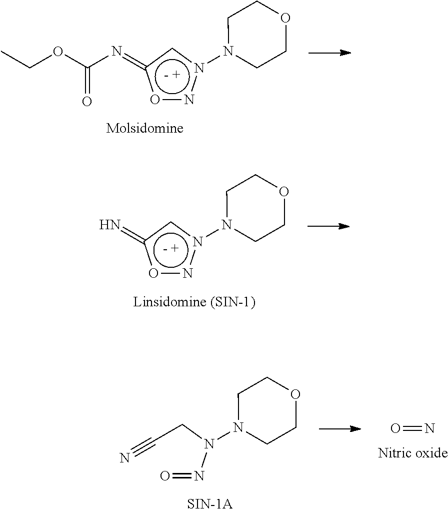

Molsidomine is a direct NO donor, that is, NO formation from molsidomine does not depend on the interactions of other substances containing thiol groups, as do nitrates.

Molsidomine is available in many forms and dosages, including:

Corvaton® (2 mg molsidomine), crospovidone (cross linked polyvinyl N-pyrrolidone, or PVP), macrogol 6000, lactose monohydrate, and magnesium stearate, available as tablets, which can be enteric coated, or ampules for IV injection;

Corvaton®-forte: 4 mg molsidomine, crospovidone, macrogol 6000, lactose monohydrate, and magnesium stearate, available as tablets, which can be enteric coated;

Corvaton®-retard: 8 mg of molsidomine, magnesium stearate, macrogol 6000, hydrogenated castor oil, microcrystalline cellulose, and lactose monohydrate (106 mg), available as tablets, which can be enteric coated.

Molsidomine also is available in an extended-release 16-mg formulation.

Molsidomine also can be includes as an active ingredient in a variety of formulations including, but not limited to, a soft patch, see, e.g., U.S. Pat. No. 4,695,465 to Kigasawa, et al., issued Sep. 22, 1987, a percutaneous pharmaceutical preparation for external use which is adapted to permit absorption of molsidomine through the skin at an optional application site without requiring oral administration or parenteral administration, see, e.g., U.S. Pat. No. 4,731,241 to Yamada, et al., issued Mar. 15, 1988; spherical granules having a core coated with spraying powder containing the active ingredient and low substituted hydroxypropylcellulose, see, e.g., U.S. Pat. No. 5,026,560 to Makino, et al., issued Jun. 25, 1991; a coating composition comprising fatty acid esters of polyglycerols, such as stearic acid penta(tetra)glyceryl ester, behenic acid hexa(tetra)glyceryl ester, lauric acid mono(deca)glyceryl ester, oleic acid di(tri)glyceryl ester, linolic acid di(hepta)glyceryl ester, palmitic acid deca(deca)glyceryl ester, and the like, see, e.g., U.S. Pat. No. 5,162,057 to Akiyama, et al., issued Nov. 10, 1992; an enteric film comprising a hydroxypropyl-methylcellulose phthalate, polyethylene glycol, and shellac, see, e.g., U.S. Pat. No. 5,194,464 to Itoh, et al., issued Mar. 16, 1993; a transdermal therapeutic composition comprising a water-soluble absorption enhancer, a fat-soluble absorption enhancer and a super water-absorbent resin, see, e.g., U.S. Pat. No. 5,362,497 to Yamada, et al., issued Nov. 8, 1994; formulations comprising spherical polyglycerol fatty acid ester granules, see, e.g., U.S. Pat. No. 5,443,846 to Yoshioka, et al., issued Aug. 22, 1995; uncoated tablets comprising an active ingredient, an excipient and an oily or fatty substance, see, e.g., U.S. Pat. No. 5,456,920 to Matoba, et al., issued Oct. 10, 1995; spherical granules having a core coated with spraying powder containing the active ingredient and low substituted hydroxypropylcellulose, see, e.g., U.S. Pat. No. 5,516,531 to Makino, et al., issued May 14, 1996; a sustained-release formulation comprising the active ingredient dispersed into a matrix comprised of a fatty acid ester of a polyglycerol, see, e.g., U.S. Pat. No. 5,593,690 to Akiyama, et al., issued Jan. 14, 1997 and U.S. Pat. No. 5,399,357 to Akiyama, et al., issued Mar. 21, 1995; a fast dissolving tablet comprising a carbohydrate and a minimum amount of water, see, e.g., U.S. Pat. No. 5,720,974 to Makino, et al., issued Feb. 24, 1998, and U.S. Pat. No. 5,501,861, to Makino, et al., issued Mar. 26, 1996; an effervescent composition, see, e.g., U.S. Pat. No. 5,824,339, to Shimizu, et al., issued Oct. 20, 1998; granules, such as those disclosed in U.S. Pat. No. 5,855,914 to Koyama, et al., issued Jan. 5, 1999; a stabilized pharmaceutical preparation comprising a tablet coated with a coating agent wherein the coating agent comprises (i) a component for the protection from light present in an amount capable of protecting the pharmaceutical from light, said component being capable of producing free radicals when exposed to ultraviolet rays, and (ii) a free radical scavenger present in an amount capable of scavenging free radicals, see, e.g., U.S. Pat. No. 6,187,340 to Fukuta, et al., issued Feb. 13, 2001; a solid pharmaceutical preparation comprising erythritol, crystalline cellulose and an disintegrants, which exhibits a fast buccal disintegratability and dissolubility, see, e.g., 6,248,357 to Ohno, et al., issued Jun. 19, 2001, and U.S. Pat. No. 5,958,453 to Ohno, et al., issued Sep. 28, 1999; a gastrointestinal mucosa-adherent matrix, see, e.g., U.S. Pat. No. 6,368,635 to Akiyama, et al., issued Apr. 9, 2002, U.S. Pat. No. 5,731,006 to Akiyama, et al., issued Mar. 24, 1998, and U.S. Pat. No. 5,593,690 to Akiyama, et al., issued Jan. 14, 1997; a solid preparation comprising one or more water-soluble sugar alcohol selected from the group consisting of sorbitol, maltitol, reduced starch saccharide, xylitol, reduced palatinose and erythritol, and low-substituted hydroxypropylcellulose, see, e.g., U.S. Pat. No. 6,586,004 to Shimizu, et al., issued Jul. 1, 2003, and U.S. Pat. No. 6,299,904 to Shimizu, et al., issued Oct. 9, 2001; a quickly disintegrating solid preparations comprising D-mannitol having an average particle size of 30 μm to 300 μm, a disintegrating agent; and celluloses, see, e.g., U.S. Pat. No. 6,740,339 to Ohkouchi, et al., issued May 25, 2004; rapidly disintegrable solid preparation comprising a sugar and a low-substituted hydroxypropylcellulose, see, e.g., U.S. Pat. No. 7,399,485 to Shimizu, et al., issued Jul. 15, 2008 and U.S. Pat. No. 7,070,805 to Shimizu, et al., issued Jul. 4, 2006; a solid pharmaceutical preparation containing a saccharide; a polyanionic polymer; a corrigent; and carboxymethylcellulose, see, e.g., U.S. Pat. No. 7,510,728 to Koike, issued Mar. 31, 2009; a controlled release capsule preparation for oral administration, see, e.g., U.S. Pat. No. 8,828,429 to Ishida, et al., issued Sep. 9, 2014; a stabilizing pharmaceutical composition, see, e.g., U.S. Pat. No. 9,186,411 to Hiraishi, et al., issued Nov. 17, 2015; an orally-disintegrating solid preparation comprising fine granules, see, e.g., U.S. Pat. No. 9,486,446 to Kurasawa, et al., issued Nov. 8, 2016, and U.S. Pat. No. 9,241,910 to Kurasawa, et al., issued Jan. 26, 2016; a tablet comprising a copolyvidone-containing coating agent, see, e.g., U.S. Pat. No. 10,098,866, to Ishida, et al., issued Oct. 16, 2018, each of which is incorporated herein by reference in its entirety.

Representative guanylate cyclase activators include, but are not limited to, 3-[2-[(4-Chlorophenyl)thiophenyl]-N-[4-(dimethylamino)butyl]-2-propenamide hydrochloride (A-350619 hydrochloride); 5-Cyclopropyl-2-[1-[(2-fluorophenyl)methyl]-1H-pyrazolo[3,4-b]pyridin-3-yl]-4-pyrimidinamine (BAY-41-2272); 2-[1-[(2-Fluorophenyl)methyl]-1H-pyrazolo[3,4-b]pyridin-3-yl]-5-(4-morpholinyl)-4 pyrimidinediamine (BAY-41-8543); 4-[[(4-Carboxybutyl)[2-[2-[[4-(2-phenylethyl)phenyl]methoxy]phenyl]ethyl]amino]methyl]benzoic acid hydrochloride (Cinaciguat hydrochloride; BAY-58-2667 hydrochloride); Guanylin; Amino-3-morpholinyl-1,2,3-oxadiazolium chloride (SIN-1 chloride); 3-(5′-Hydroxymethyl-2′-furyl)-1-benzyl indazole (YC-1), 3-Bromo-4-methyl-3,4-hexamethylene-3,4-dihydrodiazete 1,2-dioxide (DD2); and 8,13-Divinyl-3,7,12,17-tetramethyl-21H; 23H-porphine-2,18-dipropionic acid (Protoporphyrin IX).

Representative phosphodiesterase type 5 (PDE5) inhibitors include, but are not limited to, sildenafil, tadalafil, vardenafil, avanafil, mirodenafil, udenafil, lodenafil, 1-(3-Chlorophenylamino)-4-phenylphthalazine (MY-5445), 1,2-Dihydro-2-[(2-methyl-4-pyridinyl)methyl]-1-oxo-8-(2-pyrimidinylmethoxy)-4-(3,4,5-trimethoxyphenyl)-2,7-naphthyridine-3-carboxylic acid methyl ester hydrochloride (T 0156 hydrochloride), 5-[2-Ethoxy-5-[(4-ethyl-1-piperazinyl)sulfonyl]-3-pyridinyl]-3-ethyl-2,6-dihydro-2-(2-methoxyethyl)-7H-pyrazolo[4,3-d]pyrimidin-7-one benzenesulfonate (gisadenafil besylate), 2,6-bis(Diethanolamino)-4,8-dipiperidinopyrimido[5,4-d]pyrimidine (dipyridamole), 2-(2-Propyloxyphenyl)-8-azapurin-6-one (zaprinast), and cGMP Dependent Kinase Inhibitor Peptide.

In some embodiments, the presently disclosed subject matter provides a pharmaceutical composition including an agent that enhances the NO-cGMP-PKG pathway, including an NO donor, a guanylate cyclase activator, or a PDE5 inhibitor, alone or in combination with one or more additional therapeutic agents in admixture with a pharmaceutically acceptable excipient. One of skill in the art will recognize that the pharmaceutical compositions can include a pharmaceutically acceptable salt of an agent that enhances the NO-cGMP-PKG pathway. Pharmaceutically acceptable salts are generally well known to those of ordinary skill in the art and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate.

The compositions of the disclosure can be formulated for a variety of modes of administration, including systemic and topical or localized administration. Techniques and formulations generally may be found in Remington: The Science and Practice of Pharmacy (20th ed.) Lippincott, Williams & Wilkins (2000). Depending on the specific conditions being treated, such agents may be formulated into liquid or solid dosage forms and administered systemically or locally. The agents may be delivered, for example, in a timed- or sustained-slow release form as is known to those skilled in the art. Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

In yet other embodiments, the presently disclosed subject matter provides a kit comprising an NO donor. In some embodiments, the kit comprises a packaged pharmaceutical composition comprising a pharmaceutically acceptable carrier and an NO donor. The kit can further comprise indicia comprising instructions for preparing pharmaceutical compositions comprising an NO donor suitable for use with the presently disclosed methods. The kit can further comprise instructions for administering a pharmaceutical composition comprising an NO donor.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently NO donors can be administered prophylactically to prevent or reduce the incidence or recurrence of the disease, disorder, or condition.

As used herein, the term “inhibit,” and grammatical derivations thereof, refers to the ability of a presently disclosed compound, e.g., a presently disclosed compound of formula (I), to block, partially block, interfere, decrease, or reduce the growth and/or metastasis of a cancer cell. Thus, one of ordinary skill in the art would appreciate that the term “inhibit” encompasses a complete and/or partial decrease in the growth and/or metastasis of a cancer cell, e.g., a decrease by at least 10%, in some embodiments, a decrease by at least 20%, 30%, 50%, 75%, 95%, 98%, and up to and including 100%.

In general, a “therapeutically effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The terms “in combination with” or “administered in combination with” are used in their broadest sense and means that a subject is administered at least two agents, e.g., at least one nitric oxide donor and at least one anesthetic. More particularly, the terms “in combination with” or “administered in combination with” refer to the concomitant administration of two (or more) agents for the treatment of a single disease state. As used herein, the at least one nitric oxide donor and the at least one anesthetic may be combined and administered in a single dosage form, may be administered as separate dosage forms at the same time, or may be administered as separate dosage forms that are administered alternately or sequentially on the same or separate days. In one embodiment of the presently disclosed subject matter, the at least one nitric oxide donor and the at least one anesthetic are combined and administered in a single dosage form. In another embodiment, the at least one nitric oxide donor and the at least one anesthetic are administered in separate dosage forms (e.g., wherein it is desirable to vary the amount of one but not the other). The single dosage form may include additional active agents for the treatment of the disease state.

Further, the presently disclosed compositions can be administered alone or in combination with adjuvants that enhance stability of the agents, facilitate administration of pharmaceutical compositions containing them in certain embodiments, provide increased dissolution or dispersion, increase activity, provide adjuvant therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of the nitric oxide donor and the anesthetic can be varied so long as the beneficial effects of the nitric oxide donor is achieved. Accordingly, the phrases “in combination with” or “administered in combination with” refer to the administration of at least one nitric oxide donor and at least one anesthetic either simultaneously, sequentially, or a combination thereof. Therefore, a subject administered a combination of at least one nitric oxide donor and at least one anesthetic can receive the at least one nitric oxide donor and the at least one anesthetic, and optionally additional agents at the same time (i.e., simultaneously) or at different times (i.e., sequentially, in either order, on the same day or on different days), so long as the effect of the combination of the at least one nitric oxide donor and the at least one anesthetic is achieved in the subject.

When administered sequentially, the at least one nitric oxide donor and the at least one anesthetic can be administered within 1, 5, 10, 30, 60, 120, 180, 240 minutes or longer of one another. In other embodiments, when the administered sequentially, can be administered within 1, 2, 3, 4, 5, 10, 15, 20 or more days of one another. Where the at least one nitric oxide donor and the at least one anesthetic are administered simultaneously, they can be administered to the subject as separate pharmaceutical compositions, each comprising either at least one nitric oxide donor or at least one anesthetic, and optionally additional agents, or they can be administered to a subject as a single pharmaceutical composition comprising all agents. The at least one nitric oxide donor and/or the at least one anesthetic may be administered multiple times.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In particular embodiments, the subject is a neonate. As used herein, the term “neonate” or “neonatal” refers to relating to or affecting a newborn and, more particularly, a human infant during the first month, i.e., the first four weeks, including week 1, week 2, week 3, and week 4, after birth. In other embodiments, the subject is an infant, including a newborn up to a one-year old, including a 1-month, 2-month, 3-month, 4-month, 5-month, 6-month, 7-month, 8-month, 9-month, 10-month, 11-month, and 12-month old infant. In other embodiments, the subject is a 1-year to 3-year old child, including a 12-month, 13-month, 14-month, 15-month, 16-month, 17-month, 18-month, 19-month, 20-month, 21-month, 22-month, 23-month, 24-month, 25-month, 26-month, 27-month, 28-month, 29-month, 30-month, 31-month, 32-month, 33-month, 34-month, 35-month, and 36-month old child. In yet other embodiments, the subject is a fetus, including a fetus in the first trimester, the second trimester, and the third trimester of pregnancy, including the first month, the second month, the third month, the fourth month, the fifth month, the sixth month, the seventh month, the eighth month, and the ninth month of pregnancy. In even yet other embodiments, the subject is a pregnant woman, including a pregnant woman in the first trimester, the second trimester, and the third trimester of pregnancy, including the first month, the second month, the third month, the fourth month, the fifth month, the sixth month, the seventh month, the eighth month, and the ninth month of pregnancy.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### Example 1

### Nitric Oxide Donor Prevents Neonatal Isoflurane-Induced Impairments in Synaptic Plasticity and Memory

1.1 Overview

In humans, multiple early exposures to procedures requiring anesthesia is a significant risk factor for development of learning disabilities and disorders of attention and anxiety. In animal studies, newborns exposed to anesthetics develop long-term deficits in cognition. Previously work has shown that postsynaptic density (PSD)-95, discs large homolog, and zona occludens-1 (PDZ) domains may serve as molecular targets for inhaled anesthetics. The presently disclosed study investigated a role for PDZ interactions in spine development, plasticity, and memory as a potential mechanism for early anesthetic exposure-produced cognitive impairment.

In this study, postnatal day (PND) seven mice were exposed to 1.5% isoflurane (ISO) for 4 hrs or injected with 8 mg/kg active PSD-95 PDZ2WT peptide. Apoptosis, hippocampal dendritic spine changes, synapse density, long term potentiation (LTP), and cognition functions were evaluated (n=4-18).

The results from this study show that exposure of PND7 mice to ISO or PSD-95 PDZ2WT peptide causes a reduction in long thin spines (median, IQ: WT CON (0.54, 0.52 to 0.86) vs WT ISO (0.31, 0.16 to 0.38), p=0.034 and PDZ2MUT (0.86, 0.67 to 1.0) vs PDZ2WT (0.55, 0.53 to 0.59), p=0.028); impairment in LTP (median, IQ: WT CON (123, 119 to 147) and WT ISO (101, 96 to 118), p=0.0.049 and PDZ2MUT (125, 119 to 131) and PDZ2WT (104, 97 to 107), p=0.029); and deficits in acute object recognition (median, IQ: WT CON (79, 72 to 88) vs WT ISO (63, 55 to 72), p=0.044 and PDZ2MUT (81, 69 to 84) vs PDZ2WT (67, 57 to 77), p=0.039) at PND21 without inducing detectable differences in apoptosis or changes in synaptic density. Further, impairments in recognition memory and LTP were preventable by introduction of a nitric oxide (NO) donor.

Accordingly, the presently disclosed study shows that early disruption of PDZ domain-mediated protein-protein interactions alters spine morphology, synaptic function, and memory. These results support a role for PDZ interactions in early anesthetic exposure-produced cognitive impairment. Prevention of recognition memory and LTP deficits with a NO donor supports a role for the N-methyl-D aspartate (NMDA) receptor/PSD-95 PDZ2/neuronal nitric oxide synthase (nNOS) pathway in mediating these aspects of ISO-induced cognitive impairment.

1.2 Background

While pediatric anesthesia is considered safe in terms of mortality and gross morbidity, accumulating evidence suggests that early exposure to anesthetic agents may interfere with brain development, cause neuronal death, and ultimately lead to permanent cognitive deficits. Jackson et al., 2016; Gentry et al., 2013; Jevtovic-Todorovic et al., 2003; Brambrink et al., 2010; Flick et al., 2011; and Wilder et al., 2009. Recently, the United States Food and Drug Administration has identified pediatric anesthetic neurotoxicity (PAN) as a potentially important public health problem. Kuehn, 2011. Evidence from epidemiologic studies suggests humans are susceptible to long-term cognitive effects after anesthesia. Flick et al., 2011; Wilder et al., 2009; DiMaggio et al., 2009; DiMaggio et al., 2011, and Ing et al., 2012.

Multiple exposures to anesthetics before an age of three years are associated with increased frequencies of learning disabilities and attention deficit/hyperactivity disorder. Hu et al., 2017. Recent primate studies revealed persistent abnormality in visual recognition memory, Alvarado et al., 2017, and emotional reactivity, Raper et al., 2018, after early repeated anesthesia exposure. Animal models have confirmed that early postnatal exposure to anesthetics results in long-lasting impairments in learning and memory. Jevtovic-Todorovic et al., 2003; Idonomidou et al., 1999; Yon et al., 2005; Ma et al., 2007; Slikker et al., 2007; Kahraman et al., 2008; and Fredriksson et al., 2007.

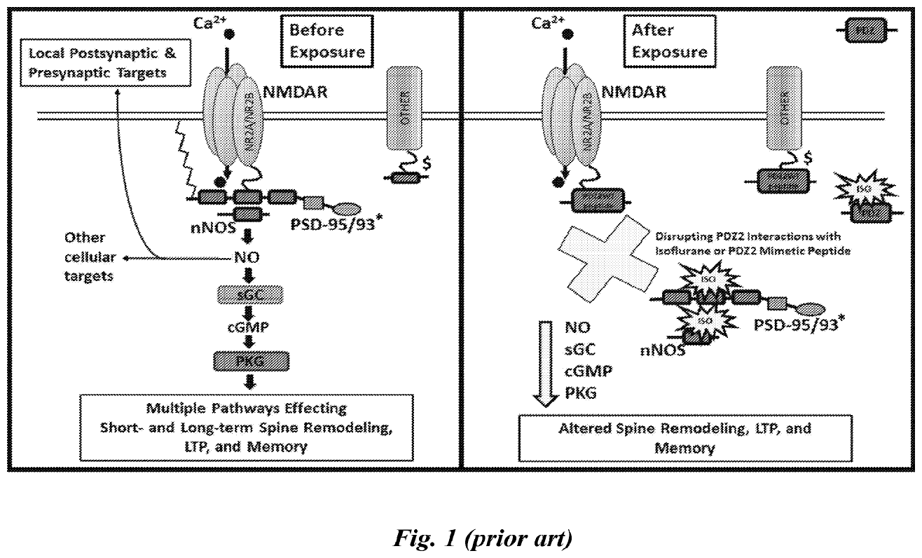

While a number of ion channels and receptors at synapses have been highlighted as potential targets for anesthetics, the molecular mechanisms that underlie PAN are still poorly understood 20-24. Franks, 2008; Solt and Forman, 2007; Campagna et al., 2003; Rudolph and Antkowiak, 2004; and Hemmings et al., 2005. Many of these ion channels and receptors are linked to their downstream signaling pathways through PDZ domain-mediated protein-protein interactions. It has been shown previously that anesthetics can disrupt PDZ domain-mediated protein-protein interactions in vitro and in vivo. Fang et al., 2003; and Tao et al., 2015. In these previous studies, PDZ domain-mediated protein interactions between PSD-95 or PSD-93 and the NMDA receptor or nNOS were dose-dependently and specifically inhibited using clinically relevant concentrations of inhalational anesthetics (FIG. 1, prior art). Fang et al., 2003. These inhibitory effects are immediate, potent, and reversible and occur at a hydrophobic peptide-binding groove on the surface of the second PDZ domain of PSD-95.

These findings revealed PSD-93 and PSD-95 proteins, and specifically their PDZ domains, as molecular targets for inhalational anesthetics. This action of anesthesia was mimicked with PSD-95 PDZ2WT peptide, which disrupts PSD-PDZ2-mediated protein interactions by binding to interaction partners (FIG. 1, prior art). More particularly, the disruption of protein-protein interactions between NMDA receptor NR2 subunits and PSD-95 was demonstrated. Tao and Johns, 2008. This disruption significantly reduced MAC and righting reflex EC50 for halothane, indicating that this domain and protein are important for anesthetic action.

Given that (1) the PDZ domain is a molecular target for inhalational anesthetics (Fang et al., 2003; Tao et al., 2015); (2) disruption of PSDPDZ2 mediated protein interactions increases anesthetic sensitivity (Tao and Johns, 2008); (3) PSD-95 PDZ2 interacts with NMDA receptor and promotes synaptogenesis (Nikonenko et al., 2008; Kornau et al., 1995); and (4) multi-innervated spine formation is prevented by deletion of the PSD-95 PDZ2 domain (Nikonenko et al., 2008), without wishing to be bound to any one particular theory, it is thought that alteration of PDZ domain-mediated protein-protein interactions contribute to the molecular mechanisms of PAN by uncoupling ion channels and receptors from their downstream signaling pathways.

As provided in more detail herein below, the presently disclosed study investigates a role for PSD-95 PDZ2 domain-mediated protein-protein interactions in hippocampal development and plasticity as a potential mechanism for early anesthetic exposure-produced cognitive impairment. More particularly, the presently disclosed subject matter examines, in vivo, the outcome of disrupting PSD-95 PDZ2 domain-mediated protein-protein interactions early in development on apoptosis, spinogenesis, synaptogenesis, long term potentiation, and object recognition memory. Further, to determine if such impairments and conditions are preventable and to specifically determine the involvement of the NDMAR NR2-PSD-95 PDZ2-nNOS pathway, a nitric oxide (NO) donor was introduced immediately following cessation of ISO anesthesia or control (O₂) exposure.

1.3. Materials and Methods

1.3.1 General Methods. This study was carried out with approval from the Animal Care and Use Committee at Johns Hopkins University and was consistent with the National Institutes of Health Guide for the Care and Use of Laboratory Animals. No surgery was performed, and all efforts were made to minimize animal suffering and reduce the number of animals used. C57BL6 wild type (WT) and PSD-93 null mutant male and female mice were used in this study. For all experiments, mice were assessed in their sexually immature state (<PND21). On PND7, animals from each litter were randomly assigned to control and treatment groups. Mice were maintained under standard lab housing with 12 h light/dark cycle. Water and food were available ad libitum until mice were transported to the laboratory approximately 1 h before the experiments.

1.3.2 Anesthesia, Peptide, and Molsidomine Injections. PND7 control and experimental mice were placed in a clear plastic cone and body temperature maintained by a heating blanket set to 35° C. Vital signs and physiological monitoring were assessed using PhysioSuite® (Kent Scientific Corporation, Torrington, Conn., USA) and blood gasses were collected arterially. These data suggested that the mice were adequately oxygenated at 100% 02 and they were not overly acidotic, that is, all mice studied had a pH of >7.2 (data not shown). Note that lower concentrations of 02 can be used, e.g., between 20% to 100% 02, including 20%, 25%, 30%, 35%, 40%, 45%, 50%, 55%, 60%, 65%, 70%, 75%, 80%, 85%, 90%, 95%, 96%, 97%, 98%, 99%, and 100% 02. Naïve animals were left with the dams. Anesthesia was initiated with 2.4% ISO in 100% oxygen for 2 min and tapered down to 1.5% within 15 min. Exposure to 1.5% ISO was continued for 3 hours and 45 min (total 4 hours ISO). Control ‘CON’ animals were exposed to 100% oxygen only. At the end of the exposure animals were maintained in oxygen on the heating blanket for 10 min then returned to dams. The purified fusion peptides, active Tat-PSD-95 PDZ2WT (also referred to as PDZ2WT herein) or inactive Tat-PSD-95 PDZ2MUT (also referred to as PDZ2MUT herein), at 8 mg/kg were injected into mice intraperitoneally (ip) in 150 μL of PBS and 10% glycerol, as previously described. Tao and Johns, 2008. A single injection of peptide was given ip. Separate peptide cohorts were injected in parallel with mice undergoing exposure to anesthesia or O₂ control. Purification of fusion peptides was performed by Creative BioMart (Shirley, N.Y.) and verified by Coomassie blue staining and Western blot analysis and then stored in 10% glycerol/phosphate buffered saline at −80° C. until use. The Tat-PSD-95 PDZ2WT and MUT plasmids used to generate proteins containing an amino-terminal, in-frame, 11-amino-acid, minimal transduction domain (residues 47-57 of human immunodeficiency virus Tat protein) termed Tat. Inactive control plasmid, mutated Tat-PSD-95 PDZ2, has three sites critical for interactions between NMDARs and PSD-95 mutated (K165T, L170R and H182L). Fang et al., 2003. The NO donor molsidomine [(N-[ethoxycarbonyl]-3-[4-morpholinosydnomine] (Sigma, St. Louis, Mo.) was injected at 4 mg/kg into mice ip in 100 μL sterile saline as previously described. The NO donor was added immediately following cessation of anesthesia or control (O₂) exposure, i.e., the NO donor was injected 4 hours after onset of anesthesia. One of ordinary skill in the art upon review of the present disclosure would appreciate that the NO donor could be administered at other times during anesthesia, including at the onset of anesthesia. Control animals were injected ip with the vehicle (saline).

1.3.3 Western Blotting. C57B16 WT mice were sacrificed by cervical dislocation and the brains were harvested. Hippocampi were grossly dissected from the mouse brain under a dissecting microscope. Total proteins from these tissues were extracted. The tissues were homogenized in homogenization buffer (10 mM Tris-HCl, 5 mM MgCl₂, 2 mM EGTA, 1 mM phenylmethylsulfonyl fluoride, 1 μM leupeptin, 2 μM pepstatin A, and 320 mM sucrose [pH 7.4]). The crude homogenates were centrifuged at 700 g for 15 min at 4° C. Then the supernatants were combined and diluted in resuspension buffer (10 mM Tris-HCl, 5 mM MgCl₂, 2 mM EGTA, 1 mM phenylmethylsulfonyl fluoride, 1 μM leupeptin, 2 μM pepstatin A, and 250 mM sucrose [pH 7.4]). Next, the protein extracts were resolved by sodium dodecyl sulfate-polyacrylamide gel electrophoresis and electrotransferred to nitrocellulose membranes. The membranes were blocked in 0.1% Tween-20 in Tris-HCl-buffered saline (TBST) containing 5% nonfat milk for 1 h at room temperature and then immunoblotted with primary antibodies (anti-caspase 3:1:1000 and poly-(adenosine diphosphate-ribose) polymerase (PARP) 1:1000 were from Cell Signaling Technology, Beverly, Mass.; anti-(3-actin: 1:100,000, Sigma-Aldrich, St. Louis, Mo.) in TBST buffer containing 5% nonfat milk overnight at 4° C. After being washed extensively in TBST, the membranes were incubated for 1 h with horseradish peroxidase conjugated anti-rabbit or anti-mouse immunoglobulin (Bio-Rad Laboratories, Hercules, Calif.) at a dilution of 1:5000. Proteins were detected by enhanced chemiluminescence (Amersham, Piscataway, N.J.). (3-Actin served as a loading control.

1.3.4 Golgi Staining, Microscopy, and Spine Reconstruction. PND21 mice were deeply anesthetized and perfused transcardially with a brief flush of 0.01 M phosphate buffered saline (pH 7.4) followed by 50 mL of 4% paraformaldehyde in 0.1 M phosphate buffer (pH 7.4). After the perfusion, the brains were removed and hippocampi were grossly dissected and stained using FD Rapid GolgiStain™ Kit, (FD NeuroTechnologies, Inc., Columbia, Md.) as per vendors instructions. Briefly, tissue was immersed in AB impregnation solution at room temperature in the dark for two weeks. Impregnation solution was replaced after the first overnight on the next day. Tissue was transferred to solution C for 72 hours. Hippocampi were embedded in tissue freezing medium (TFM) and stored at −80° C. 60-μm sections were cut on a cryostat at −20° C., mounted onto gelatin coated slides, and air dried overnight. Slides were rinsed in Milli-Q water, developed in the working solution DE for 10 min, rinsed, dehydrated in EtOH, cleared in xylene, and mounted with Permount®. Only samples that had optimal impregnation (contiguous staining across dendrites and spines) were taken forward for imaging (n=2 excluded animals).

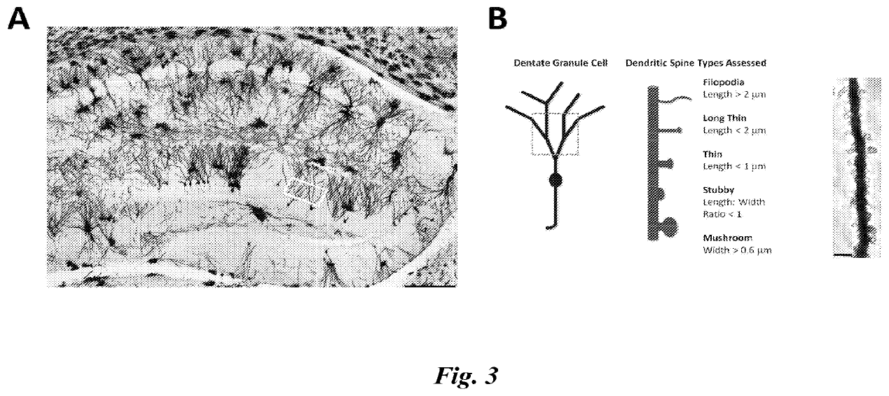

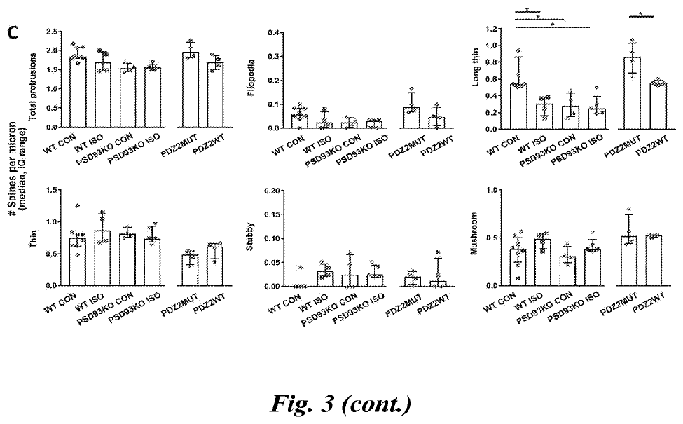

Two different imaging fields per mouse each containing at least three unique dendritic segments (six segments total per mouse), which contain dorsal hippocampus were imaged. The six segments were averaged per mouse to contribute one data point per mouse. Dentate granule cells were identified by their location within the dentate gyrus and their distinct morphology. Spines along secondary and tertiary dendrites of these neurons were selected for analysis (see FIG. 3). Z-stacks of Golgi stained dendrites (optical section thickness=0.3 μm, i.e., 50 to 100 images per stack) were taken at 630× magnification on a Leica SPE confocal microscope. Spine analysis was performed as described in Risher et al., 2014, using the freely available RECONSTRUCT software. Fiala, 2005.

1.3.5 Electron Microscopy. PND21 mice were deeply anesthetized and perfused transcardially with a brief flush of 35° C. Mammalian Ringer Solution (EMS 11763-10) with 5 U/mL of Heparin (Sagent Pharmaceuticals NDC 25021-400-10) followed by 50 mL of 2.5% paraformaldehyde (freshly prepared from EM grade prill), 2% glutaraldehyde, 0.1M sodium cacodylate, 3 mM MgCl₂, (pH 7.2) at 1314 mOsmols at a rate of 1-ml/min. Animals were administered a lethal dose of isoflurane (5% isoflurane in oxygen until respiration ceases). The animals were checked for hind paw pinch withdrawal and eye blink reflexes to confirm complete anesthesia. The chest cavity was then cut open to expose the heart for perfusion, and the resultant pneumothorax ensured rapid lethality. After the perfusion, the mouse heads were stored at 4° C. for 2 hours. After incubation in the cold, brains were removed and hippocampi were grossly dissected using a brain block and dissecting microscope. Tissue was immersed in fixative overnight and further dissected in the cold room the next morning to isolate the hippocampus (2 mm×2 mm) and add notches for orientation. The following steps were kept cold (4° C.) until the 70% ethanol step, then run at room temp. Samples were rinsed in 100 mM cacodylate 3.5% sucrose 3 mM MgCl₂, pH 7.2 at 324 mOsmols for 45 min. Following buffer rinses, samples were microwave fixed twice in 2% osmium tetroxide reduced with 1.6% potassium ferrocyanide, in the same buffer without sucrose. Sample temperatures did not exceed 9° C. Following microwave processing, samples were rocked in osmium on ice for 2 hours in the dark. Tissue was then rinsed in 100 mM maleate buffer with pH 6.2, then en-bloc stained for 1 hour with filtered 2% uranyl acetate in maleate buffer, pH 6.2. Following en-bloc staining samples were dehydrated through a graded series of ethanol to 100%, transferred through propylene oxide, embedded in Eponate 12 (Pella) and cured at 60° C. for two days. Sections were cut on a Riechert Ultracut E microtome with a Diatome Diamond knife (45 degree). 60-nm sections were picked up on formvar coated 1-mm×2-mm copper slot grids and stained with methanolic uranyl acetate. Grids were viewed on a Phillips CM 120 TEM operating at 80 kV and digital images captured with an XR80 8-megapixel CCD by AMT. Ten images (each image 6250 nm×7500 nm) were averaged per animal. Each animal contributed one data point to obtain median number of PSD's for each group.

1.3.6 Electrophysiology.

1.3.6.1. Slice preparation. Two weeks after exposure (PND21-35) mice were euthanized and coronal brain slices containing central part of hippocampus (300 μm thick) were made from Leica VT 1200S vibrotome in ice cold ACSF containing: 128 mM NaCl, 3 mM KCl, 26 mM NaHCO₃, 1 mM NaH₂PO₄, 1 mM MgSO₄, 10 mM glucose and 2 mM CaCl₂ and saturated with 95% 02 and 5% CO₂. The slices were incubated for at least 1 hr at room temperature (22° C.-24° C.) in the interface-type holding chamber filled with ACSF. Then a slice was transferred to the recording chamber, where ACSF was perfused at a rate of 1.5-2.0 mL/min at room temperature.

1.3.6.2. Extracellular field-potential recordings. Synaptic responses were recorded using a MultiClamp 700B amplifier, and the signal was digitized with Digidita 1440A, analyzed with pClamp10 and stored on a personal computer. Extracellular recordings of field excitatory postsynaptic potentials (fEPSPs) were made from the stratum radiatum of the hippocampal CA1 area. Evoked responses were elicited with 0.1-msec constant-current pulses through a concentric electrode in the Schaffer collateral pathway every 30 sec at an intensity sufficient to elicit 40-50% maximal EPSPs. After establishing a stable baseline for 20 min, LTP was induced by applying three trains of 100 Hz×1 sec high frequency stimulus (HFS) 20 sec apart at the baseline stimulus intensity. Measurements of the fEPSP slopes were made during the rising phase (5-50% of the peak) and the values normalized to the mean values recorded in 20-min baseline. The median of normalized fEPSP slopes 55-60 min after HFS was used for comparison between groups. Each animal contributed one data point to obtain the median for each group.

1.3.7 Novel Object Recognition. The novel object recognition (NOR) procedure was based upon the original Ennaceur et al., 1988, procedure and was used to assess nonspatial hippocampal memory. Clark et al., 2000; Baker and Kim, 2002; Reger et al., 2009. It consisted of a training ‘familiarization’ phase followed by a testing phase. During training, mice were allowed to freely explore within an opaque box (40 cm W×40 cm L×34 cm H) containing two identical objects for 10 min. Data were recorded with a video camera and time spent with each object was recorded using ANYmaze software (Stoelting). Object investigation time was determined by the amount of time the mouse spent in the zone immediately surrounding the object. Only mice that investigated the objects for at least 10 sec (criterion) were taken forward to the testing phase (n=6 animals were not taken forward as they did not meet criterion). After 2 h, object recognition was tested, using the same procedure as in training except that a novel object was substituted for one of the familiar training objects and mice were allowed to explore for 5 min. Mice inherently prefer to explore novel objects; thus, a preference for the novel object indicates intact memory for the familiar object.

1.3.8 Statistical Analysis. Statistical analysis was carried out by unpaired Student's t-test, two-tailed, Mann-Whitney, and Kruskal-Wallis followed by post-hoc Dunn's tests with Graphpad Prism version 7.0 software (Graphpad Inc., La Jolla, Calif.). Datasets that failed D'Agostino & Pearson normality test were analyzed using non-parametric statistics. Student's t-test, two-tailed was used to compare novel and known object investigation times in the NOR assay.

Mann-Whitney, two-tailed test was used to compare WT CON versus WT ISO and PDZ2MUT versus PDZ2WT groups (PSD quantification, NOR+NO donor, and LTP+NO donor). Mann-Whitney, two-tailed test was used to compare WT CON versus WT ISO groups (PARP WB) and PDZ2MUT versus PDZ2WT groups (spine analysis and LTP). Kruskal-Wallis test was used in analysis of spines and LTP (comparing WT CON, WT ISO, PSD93KO CON, PSD93KO ISO which included one family, four treatments, and six comparisons) and NOR (comparing WT NAÏVE, WT CON, WT ISO, PSD93KO CON, PSD93KO ISO which included one family, five treatments and ten comparisons) and (PDZ2MUT, PDZ2WT, PDZ2WT+ISO that include one family, three treatments, and three comparisons). Data are expressed as mean±standard deviation (SD) or median, interquartile range respectively, and statistical significance was set at P<0.05. *p<0.05, **p<0.01, ***p<0.001, and ****p<0.0001.

Sample sizes were chosen based on previous experience and/or published literature. A sample size of N=6 was chosen for PARP WB because an N=3 was sufficient to detect significant differences following forebrain injury in rats. Taylor et al., 2006. A sample size of N=3 to 6 was chosen for ultrastructure analysis because this number of animals was sufficient to detect significant differences in PSDs in the DG after cerebral ischemia. Martone et al., 1999. A sample size of N=4 to 8 was chosen for spine analysis because an N=4 was sufficient to show differences in hippocampal proportional densities of different types of spines following excitoxicity. Gonzalex-Burgos et al., 2009. A sample size of N=4 to 7 was chosen for LTP measurements because an N=3 was sufficient to show differences in LTP amplitude induced by NMDA antagonist. Pigott and Garthwaite, 2016. A sample size of N=6 to 18 was chosen for NOR because sample sizes of 10 were sufficient to show age specific differences in rodents. Reger et al., 2009; Benice et al., 2006. In all experiments each animal contributed one data point to obtain the median for each group.

1.4 Results

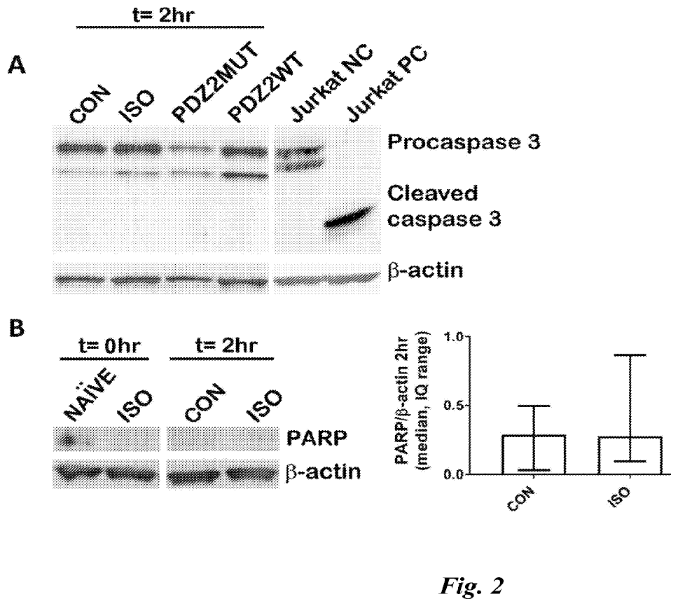

1.4.1 Effect of a single dose of ISO or PDZ2WT peptide on apoptosis. Jurkat cell control protein obtained from cell signaling technology was run to determine parameters for running Caspase-3 Western blot. The negative control was generated by lysing in Chaps cell extract buffer and positive control was treated with cytochrome C. A representative image shows expected uncleaved (procaspase 3) and cleaved (cleaved caspase 3) bands (FIG. 2A). 4 h treatment in mice including ISO or PDZ2WT peptide exposure did not yield any detectable cleaved caspase at 2 h following cessation of exposure (FIG. 2A). In this experiment, isoflurane exposure was given in 100% 02 (1.5% ISO in 100% 02). Peptide injected animals were not placed in oxygen. They received a single ip injection. The control for the ‘active’ peptide (PDZ2WT) was a mutant form ‘inactive’ peptide (PDZ2MUT). Cleaved caspase 3 was not detected at 0 h or 20 h following cessation of exposure (data not shown). Representative image shows expected cleaved PARP band (FIG. 2B left). 4 h ISO treatment in mice did not yield significantly different levels of PARP compared to CON at 2 h following cessation of exposure (FIG. 2B right, median, interquartile range: CON (0.29, 0.31 to 0.50) vs ISO (0.28, 0.09 to 0.87), p=0.818). Data were analyzed using Mann-Whitney test.

1.4.2 ISO, PSD93 deficiency, and PDZ2WT peptide alter hippocampal dendritic spine morphology. To determine whether inhaled anesthetics interfere with spinogenesis by disrupting synaptic PDZ interactions in the developing hippocampus, the impact of ISO, PSD-93 deficiency, and disrupting PSD-95 PDZ2 domain-mediated protein-protein interactions on dendritic spine morphology in PND21 mice two weeks following an exposure at PND7 was investigated. Of note, the PSD-95 family (PSD-95, PSD-93, SAP102, SAP97) of membrane associated guanylate kinases (MAGUKs) share high sequence similarity, as well as similar domain structure: three PDZ domains followed by an SH3 and a GK domain. Funke et al., 2005; Sheng and Hoogenraad, 2007. PDZ domains of PSD93 are remarkably similar to the PDZ domains of PSD95 in sequence and structure. Fiorentini et al., 2009.

Both PSD-93 and PSD-95 PDZ domain-mediated interactions with NMDA NR2 or nNOS can be disrupted with anesthetics (FIG. 1). Fang et al., 2003. Since PSD-93 KO mice show impaired LTP, Carlisle et al., 2008, similar to anesthesia exposed WT rodents, Jevtovic-Todorovic et al., they were used herein as a representative global knockout for the PSD-95 family of MAGUKs in assessments on spine morphology, LTP induction, and memory. Neonatal WT and PSD93KO mouse pups (PND7) were exposed to ISO or 02 for 4 hrs. A separate cohort of WT animals also was exposed to PDZ2MUT or PDZ2WT peptides. Animals were harvested two weeks later (PND21) for rapid Golgi staining to visualize hippocampal dendritic spines (FIG. 3). An example tiled image of a Golgi stained region of the hippocampus is shown in FIG. 3A. The white box indicates the subregion of interest within the superior blade of the DG. The total number of protrusions and different spine types were assessed based on dendritic segments distal to the first and second branch points (FIG. 3B). Data were analyzed using Kruskal-Wallis and Mann Whitney tests.

A Kruskal-Wallis test indicated a main effect on the number of total dendritic protrusions across the following four groups (FIG. 3C top left, median, IQ: WT CON (1.8, 1.8 to 2.1), WT ISO (1.7, 1.5 to 2.0), PSD93KO CON (1.5, 1.5 to 1.7), PSD93KO ISO (1.6, 1.5 to 1.6), p=0.029. No difference, however, was detected following Dunn's multiple comparison tests (WT CON vs WT ISO, p=0.766; WT CON vs PSD93KO CON, p=0.329; WT CON vs PSK93KO ISO, p=0.094; WT ISO vs PSD93KO CON, p>0.999; WT ISO vs PSD93KO ISO, p>0.999; PSD93KO CON vs PSD93KO ISO, p>0.999). No significant effect on total protrusions was observed between PDZ2MUT (2.0, 1.8 to 2.2) vs PDZ2WT (1.7, 1.5 to 1.9), p=0.114. Thus, exposure to ISO, PSD93 deficiency, or PDZ2WT peptide did not have a significant effect on total number of dendritic protrusions assessed at PND21.

ISO, PSD93 deficiency, or PDZ2WT peptide did not have significant effects on the number of filopodial type protrusions (length>2 μm) (FIG. 3C top middle, WT CON (0.06, 0.04 to 0.08), WT ISO (0.02, 0.00 to 0.07), PSD93KO CON (0.02, 0.00 to 0.05), PSD93KO ISO (0.03, 0.00 to 0.03), p=0.062. PDZ2MUT (0.09, 0.07 to 0.15) vs PDZ2WT (0.05, 0.01 to 0.09), p=0.200).

A main effect of treatment was observed across the following groups on the number of long thin spines (<2 μm length and <0.6 μm width) (FIG. 3C top right, WT CON (0.54, 0.52 to 0.86), WT ISO (0.31, 0.16 to 0.38), PSD93KO CON (0.28, 0.16 to 0.44), PSD93KO ISO (0.25, 0.19 to 0.39), p=0.003. ISO caused a reduction in long thin spines in WT mice as did PSD93 deficiency (WT CON vs WT ISO, p=0.034; WT CON vs PSD93KO CON, p=0.042; WT CON vs PSD93KO ISO, p=0.015; WT ISO vs PSD93KO CON, p>0.999; WT ISO vs PSD93KO ISO, p>0.999). ISO did not further reduce the number of long thin spines in PSD93 KO mice (PSD93KO CON vs PSD93KO ISO, p>0.999). PDZ2WT peptide caused a reduction in long thin spines (PDZ2MUT (0.86, 0.67 to 1.0) vs PDZ2WT (0.55, 0.53 to 0.59), p=0.028.

ISO, PSD93 deficiency, or PDZ2WT peptide did not have a significant effect on number of thin spines (length<1 μm) (FIG. 3C bottom left, WT CON (0.75, 0.62 to 0.83), WT ISO (0.87, 0.68 to 1.13), PSD93KO CON (0.82, 0.76 to 0.91), PSD93KO ISO (0.74, 0.68 to 0.93), p=0.705; PDZ2MUT (0.49, 0.34 to 0.54) vs PDZ2WT (0.62, 0.43 to 0.66), p=0.200).

ISO, PSD93 deficiency, or PDZ2WT peptide did not have a significant effect on stubby spines (length:width ratio<1) (FIG. 3C bottom middle, WT CON (0.00, 0.00 to 0.00), WT ISO (0.03, 0.02 to 0.05), PSD93KO CON (0.02, 0.00 to 0.07), PSD93KO ISO (0.02, 0.02 to 0.04), p=0.051; PDZ2MUT (0.02, 0.00 to 0.03) vs PDZ2WT (0.01, 0.00 to 0.06), p=0.914).

ISO, PSD93 deficiency, or PDZ2WT peptide did not have a significant effect on number of mushroom type protrusions (FIG. 3C bottom right, WT CON (0.38, 0.25 to 0.50), WT ISO (0.49, 0.38 to 0.54), PSD93KO CON (0.31, 0.24 to 0.41), PSD93KO ISO (0.38, 0.36 to 0.48), p=0.283; PDZ2MUT (0.52, 0.44 to 0.74) vs PDZ2WT (0.53, 0.51 to 0.53), p>0.999).

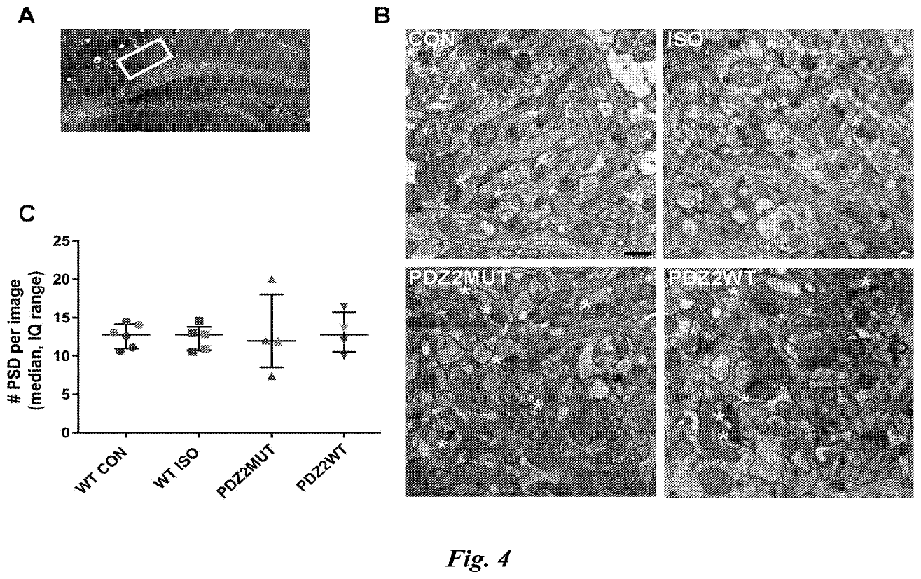

1.4.3 ISO and PDZ2WT peptide do not have an effect on the number of postsynaptic densities in the hippocampus at PND21. To investigate the impact of neonatal exposure to ISO or disruption of PSD-95 PDZ2 domain-mediated protein-protein interactions on synaptogenesis, the number of PSDs in the hippocampus in PND21 mice were assessed two weeks after exposure (FIG. 4). Neonatal exposure to ISO or PDZ2WT peptide did not have a significant effect on number of PSDs as compared to controls (FIG. 4C, median, IQ: WT CON (12.8, 10.9 to 14.1) vs WT ISO (12.9, 10.7 to 13.8), p=0.829; PDZ2MUT (12.0, 8.5 to 18) vs PDZ2WT (12.8, 10.5 to 15.7), p=0.743). Data were analyzed using Mann-Whitney test.

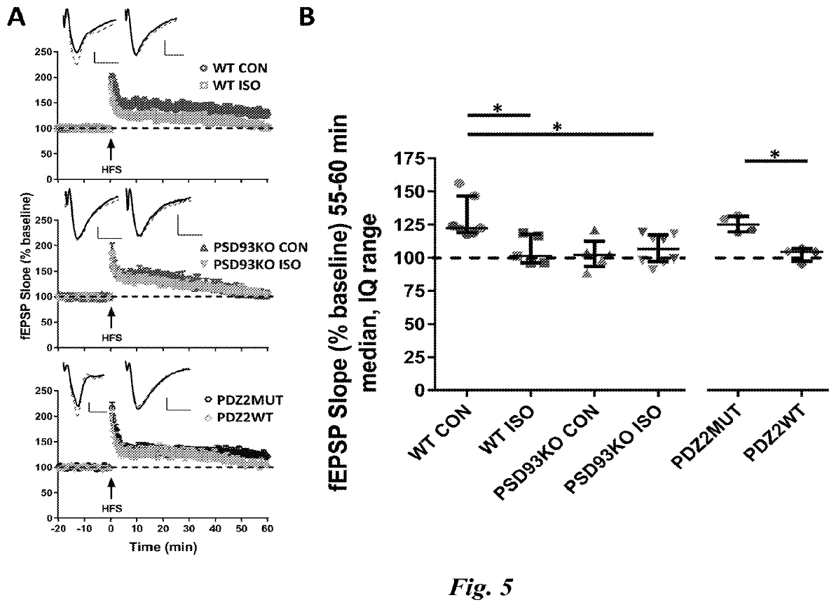

1.4.4 ISO, PSD93 deficiency, and PDZ2WT peptide impair LTP induction in hippocampal CA1 at PND21. To assess long-term electrophysiological effects of early ISO exposure, PSD-93 deficiency, and disruption of PSD-95 PDZ2 domain-mediated protein-protein interactions, synaptic function and LTP were examined in hippocampal slices prepared at PND21 from mice treated at PND7. Two weeks after exposure, robust LTP can be induced in WT mice that received oxygen CON exposure (FIG. 5A top, FIG. 5B; median, IQ: 123, 119 to 147) or inactive PDZ2MUT peptide (FIG. 5A bottom, FIG. 5B; 125, 119 to 131) 55-60 min after HFS. Expression of LTP was impaired in ISO (FIG. 5A top, FIG. 5B; WT ISO,101, 96 to 118; PSD93KO ISO,107, 97 to 117), PDZ2WT peptide (FIG. 5A bottom, 5B; 104, 97 to 107) and PSD93KO CON (FIG. 5A middle, FIG. 5B; 102, 94 to 112) groups. Kruskal-Wallis test indicated a significant effect of treatment (WT CON, WT ISO, PSD93KO CON, PSD93 ISO, p=0.009). Post-hoc Dunn's analysis showed significant differences between WT CON vs WT ISO (FIG. 5B; p=0.049) and WT CON vs PSD93KO ISO (FIG. 5B; p=0.026). No differences were observed in the following comparisons (WT CON vs PSD93KO CON, p=0.056; WT ISO vs PSD93KO CON, p>0.999; WT ISO vs PSD93KO ISO, p>0.999; PSD93KO CON vs PSD93KO ISO, p>0.999). PDZ2MUT vs PDZ2WT (FIG. 5B; p=0.029) groups differed during the last 5 min of recording after HFS.

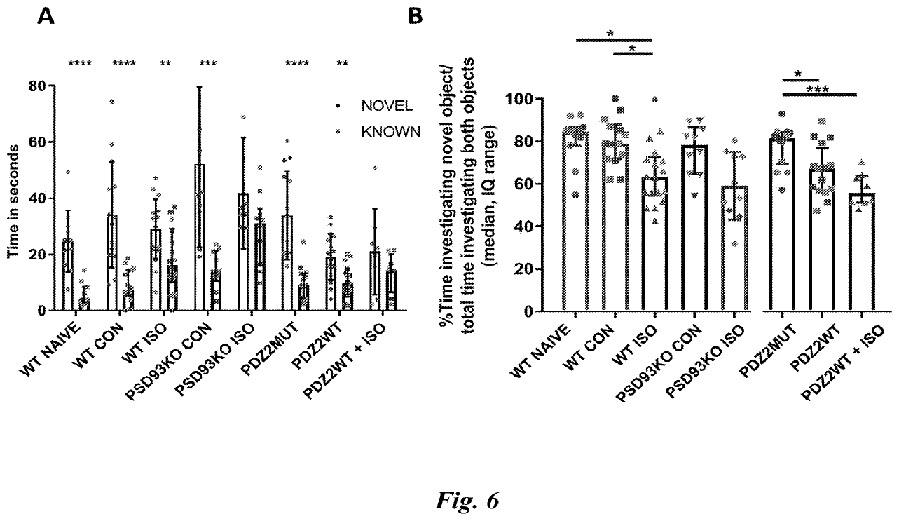

1.4.5 ISO and PDZ2WT peptide cause subtle but significant decreases in recognition memory in the novel object recognition test. To determine whether the disruption of synaptic PDZ interactions contributes to cognitive impairment after early anesthetic exposure, the impact of ISO, PSD93 deficiency, and disrupting PSD-95 PDZ2 domain-mediated protein-protein interactions on memory was investigated by assessing hippocampal dependent object recognition in PND21 mice two weeks after exposure at PND7. In the majority of conditions, mice were able to discriminate at some level between novel and known objects, revealed by increased investigation times of the novel object (FIG. 6A; mean, ±SD: WT Naïve (25±11 vs 6±4), p<0.0001; WTCON (34±19 vs 9±6), p<0.0001; WT ISO (29±11 vs 18±12), p=0.005; PSD93KOCON (52±30 vs 15±7), p=0.001; PDZ2MUT (34±16 vs 10±6), p<0.0001; PDZ2WT (19±8 vs 10±6), p=0.001; two-tailed t-test novel vs. known). The double hit animals were unable to significantly discriminate between novel and known objects (PSD93KO ISO (42±20 vs 29±13), p=0.098; PDZ2WT+ISO (21±15 vs 14±7), p=0.227). All groups spent more than 50% of their object interaction time with the novel object as can be seen in the recognition index (% time investigating novel object over time investigating novel object plus familiar object×100) plot (FIG. 6B). Kruskal-Wallis test indicates a significant effect of exposure treatment (FIG. 6B; median, IQ: WT NAÏVE (85, 78 to 87), WT CON (79, 72 to 88), WT ISO (63, 55 to 72), PSD93KO CON (78, 65 to 87), PSD93KO ISO (57, 47 to 73), p=0.0006). Post-hoc Dunn's comparisons across groups shows that ISO-exposed WT animals have a subtle but significant decrement in recognition memory as compared to controls (WT NAÏVE vs WT ISO, p=0.023 and WT CON vs WT ISO, p=0.0.044). PSD93 deficiency did not have a significant effect on recognition memory (WT NAÏVE vs PSD93KOCON, p>0.999 and WT CON vs PSD93KOCON, p>0.999). ISO exposure did not cause detectable impairment in PSD93 deficient animals (PSD93KO CON vs PSD93KO ISO, p=0.177). Kruskal-Wallis test indicates a significant effect of peptide treatment on memory (PDZ2MUT (81, 69 to 84), PDZ2WT (67, 57 to 77), PDZ2WT+ISO (56, 51 to 64), p=0.001). PDZ2WT exposed animals have a subtle but significant decrement in recognition memory as compared to PDZ2MUT controls (PDZ2MUT vs PDZ2WT, p=0.0.039 and PDZ2MUT vs PDZ2WT+ISO, p=0.001). ISO did not cause further significant decrement in recognition memory in PDZ2WT exposed animals (PDZ2WT vs PDZ2WT+ISO, p=0.386).

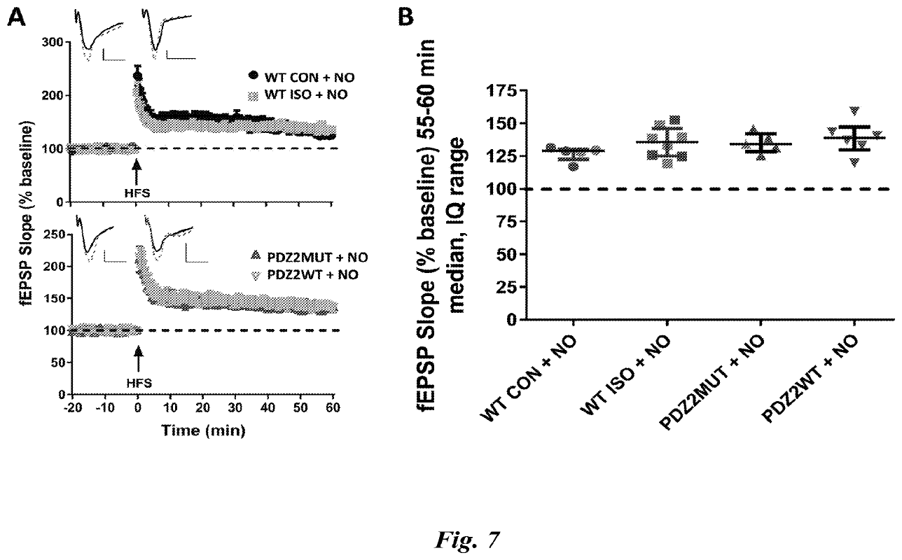

1.4.6 Treatment with NO donor prevents the negative effects of ISO and PDZ2WT peptide on hippocampal LTP. Treatment with NO donor prevents the impairment in LTP caused by ISO or PDZ2WT peptide as indicated by the renewed expression of LTP (FIG. 7A, FIG. 7B; median, IQ: WT CON+NO (129, 123 to 130), ISO+NO (136, 125 to 146), PDZ2MUT+NO (134, 128 to 142), PDZ2WT+NO (139, 130 to 147). There is no longer a significant difference between WT CON and WT ISO when NO donor is added (FIG. 7B; WT CON+NO vs WT ISO+NO, p=0.284) or between PDZ2MUT and PDZ2WT (FIG. 7B; PDZ2MUT+NO vs PDZ2WT+NO, p=0.662). Data were analyzed with Mann-Whitney test.

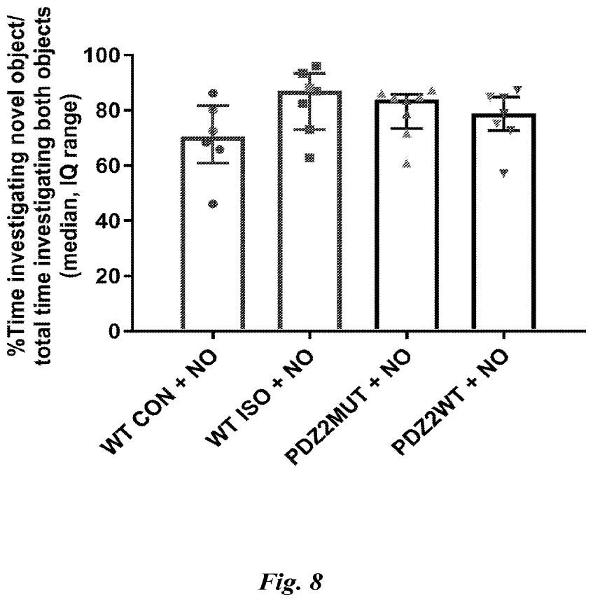

1.4.7 Treatment with NO donor prevents ISO- or PDZ2WT-induced impairment in acute recognition memory. Treatment with NO donor prevents the impairment in NOR caused by ISO or PDZ2WT peptide as indicated by the increased discrimination in NOR (FIG. 8; median, IQ: WT CON+NO (71, 61 to 82), WT ISO+NO (87, 73 to 93), PDZ2MUT+NO (84, 73 to 86), PDZ22WT+NO (79, 73 to 85). There is no longer a significant difference between WT CON and WT ISO when NO donor is added (WT CON+NO vs ISO+NO, p=0.073) or between PDZ2MUT and PDZ2WT (PDZ2MUT+NO vs PDZ2WT+NO, p=0.779). Data were analyzed with Mann-Whitney test.

1.5 Discussion

To better understand the mechanisms mediating ISO-induced cognitive impairment one specific molecular target of anesthesia was investigated. It had been previously demonstrated that inhalational anesthetics can disrupt PDZ domain-mediated protein-protein interactions in vitro and in vivo, Fang et al., 2003; Tao et al., 2015, and specifically inhibit the PDZ domain-mediated protein interaction between PSD-95 or PSD-93 and the NMDA receptor NR2 subunits or nNOS (FIG. 1). Fang et al., 2003; Tao and Johns, 2008. Herein, the effects of this disruption in vivo were determined in the context of ISO exposure by specifically mimicking this isolated action of anesthesia with the PDZ2WT peptide, which disrupts PSD-PDZ2-mediated protein interactions by binding to interaction partners, such as NMDA receptor NR2. In contrast to earlier work, Jevtovic-Todorovic et al., 2002, it was found that neither ISO exposure nor PDZ2WT peptide induced a detectable increase in the level of apoptosis (FIG. 2). These findings, however, are consistent to those of others, which showed brief exposures to anesthesia did not induce apoptosis. Zhu et al., 2010; Kodama et al., 2011; and Briner et al., 2010.

Further investigation into the sublethal effects of ISO, PSD-93 deficiency, and disruption of PSD-95 PDZ2 domain-mediated protein-protein interactions revealed changes in spine morphology, impairments in LTP induction, and impairments in memory. Introduction of a NO donor at the time of exposure prevents impairment in LTP and recognition memory, further implicating the involvement of the NMDAR NR2-PSD-95 PDZ2-nNOS signaling pathway in early anesthetic exposure-produced cognitive impairment.

Alterations in spine number and shape are associated with cognitive and developmental dysfunction in various neurological disorders. Blanpied and Ehlers, 2004. Anesthesia exposure, nNOS activity, and modulation of MAGUK levels have all been associated with spine morphological and density changes. Nikonenko et al., 2008; Vickers et al., 2006; and Nikonenko et al., 2002. The most striking result in the presently disclosed spine analysis is the significant loss of long thin spines (length<2 microns and width<0.6 microns) in ISO-exposed, PSD-93 null mutants, and PDZ2WT peptide exposed animals compared to controls that persists 14 days after exposure. Others also have shown selective loss of spines from anesthesia, such as the persistent decrease (up to 90 days) in spine density of spines with head diameter between 0.3-0.4 microns. Briner et al., 2011. Length-shortening of dendritic protrusions following sevoflurane anesthesia, Zimering et al., 2016, and reduction of long protrusions following ISO anesthesia, Head et al., 2009; Lemkuil et al., 2011, in culture have been reported. Platholi specifically showed that ISO reduces F-actin concentration in spines, suggesting a role in spine shrinkage and loss. Platholi et al., 2014. Thus, the loss of long thin spines observed in the presently disclosed study could be the result of length shortening following disruption of PDZ-domain mediated interactions that lead to F-actin depolymerization. The functional significance of this loss of long thin spines remains to be determined but might involve a role in circuitry development and permanently alter neural connectivity.

Anesthesia-induced changes in dendritic spine density have been shown to be accompanied by parallel changes in spine synapse number. Briner et al., 2011; Head et al., 2009. No changes in density of synapses were observed following ISO or PDZ2WT peptide exposure. The presently disclosed results demonstrate that exposure of immature mice to a sublethal dose of anesthesia or PDZ2WT peptide at the peak of synaptogenesis did not cause significant differences in hippocampal ultrastructural synaptic density two-weeks later at PND21. These findings are consistent with the lack of synaptic density change in the single 2-h anesthesia exposure observed in the Amrock study, Amrock et al., 2015, and lack of synaptic density loss in PSD-95 null mutants. Miguad et al., 1998. Follow up studies are underway to determine whether synaptic density changes occur following the presently disclosed exposure paradigm in mice allowed to recover for longer periods (i.e., greater than PND21). To further explore whether the disruption of synaptic PDZ interactions could contribute to learning and memory deficits through altered synaptic function after early anesthetic exposure, LTP, a widely considered major cellular mechanism underlying learning and memory, was investigated. Cooke and Bliss, 2006. Previous work demonstrated early exposure to a combination anesthetic induced a profound suppression of LTP in the hippocampus of adolescent rats. Jevtovic-Todorovic et al., 2003. The presently disclosed study found suppression of LTP in ISO-exposed WT animals. Similar to Carlisle et al., 2008, LTP was found to be impaired in PSD93 mutant mice. Like ISO-exposed animals PDZ2WT peptide exposed mice showed impaired LTP as compared to controls. Thus, disrupting PDZ domain-mediated protein interactions mimicked the effect of ISO on LTP. These results suggest synaptic PDZ interactions may contribute to anesthesia-induced impairment in synaptic function in mice and therefore could contribute to learning and memory deficits.

Jevtovic-Todorovic et al. first reported persistent impairments in learning and memory following early exposure to anesthetics in rats over a decade ago. Jevtovic-Todorovic et al., 2003. Subsequent studies have linked early exposure to anesthesia to impairments on hippocampal-dependent recognition memory tests in rodents, Zhu et al., 2010; Shih et al., 2012; Stratmann et al., 2014(a); Stratmann et al., 2009 and humans. Stratmann et al., 2014(b). Thus, without wishing to be bound to any one particular theory, it was thought that if disruption of synaptic PDZ interactions contributes to impairments in learning and memory after early anesthetic exposure, then deficits in recognition memory performance should be observed. The presently disclosed study found WT mice exposed to ISO exhibited reduced recognition memory performance compared to controls, as was expected. Injection of PDZ2WT peptide mimicked the effect of ISO reducing recognition memory performance as compared to inactive peptide. These results indicate that intact PSD-PDZ2-mediated protein interactions are important for hippocampal dependent recognition memory performance in weanling mice.

Several PDZ domain-mediated protein-protein interactions linking ion channels and receptors to their downstream signaling pathways have been shown to be disrupted by inhalational anesthetics. It has previously been demonstrated that PDZ domain-mediated interactions between PSD-95 or PSD-93 and NMDA receptors or nNOS, Fang et al., 2003, PSD-95 and Shaker-type potassium channel Kv1.4, Tao et al., 2015, and between AMPAR subunit GluA2 and its interacting proteins-glutamate receptor interacting protein or protein interacting with c kinase 1, Tao et al., 2015, are disrupted by clinically relevant concentrations of anesthetics. Again, without wishing to be bound to any one particular theory, it was thought that introduction of certain downstream signaling components during the time of ISO or PDZ2WT peptide exposure may prevent deficits in LTP and memory. Indeed, treatment with the NO donor, molsidomine, prevents impairment in LTP and recognition memory caused by ISO or PDZ2WT peptide, suggesting the effect of NO is downstream of the disrupted PDZ interactions. These results support the involvement of the NMDAR NR2-PSD-95 PDZ2-nNOS signaling pathway in ISO-mediated impairment in LTP and recognition memory. A better understanding of how NO signaling affects changes in LTP and recognition memory in this developmental context is an important area to pursue.

This study was limited in several important respects. Animals were not phenotyped on noncognitive behaviors so other aspects of neurodevelopment is Undefined here. In vivo evidence suggests that hyperoxia may be harmful to developing neurons, Felderhoff-Mueser et al., 2004, and there also is the potential for enhanced toxicity by combining an inhalational anesthetic with 100% oxygen so the use of 100% oxygen as a carrier gas may be questioned. This procedure is unlikely to explain the presently disclosed results, however, because control mice received 100% oxygen for the same period of time and peptide injected mice were not exposed to the oxygen carrier gas or to anesthetic, and showed parallel results. The number of animals in the presently, disclosed spine and synapse analyses are small. To reduce the effect of a small “N” multiple measurements were taken per animal in an effort to lower variation (each animal provided one data point; six dendritic fields were averaged per animal in the spine analysis and ten images were averaged per animal in the EM analysis). The ultrastructural in this study resolution was not high enough to permit length and width assessments on PSDs.

1.6 Summary

The presently disclosed subject matter indicates that a single 4-hr exposure of infant mice to ISO (approximately one MAC) or targeted disruption of PSD-PDZ2-mediated protein interactions (i.e., a specific molecular target of ISO) with PDZ2WT peptide results in spine morphological changes, impairments in LTP induction, and impairments in memory in mice without inducing apoptosis or changes in synaptic density. The observed impairments in LTP and object recognition memory can be prevented by introduction of an NO donor suggesting the involvement of NMDAR NR2-PSD-95 PDZ2-nNOS signaling pathway in these processes.

### Example 2

### Neonatal Disruption of Postsynaptic Density Protein-95 Protein Interactions Mediates Anesthetic Induced Changes in Dendritic Spines and Cognitive Function in Adult Mice

2.1 Overview

In humans, multiple early exposures to procedures requiring anesthesia is a significant risk factor for development of learning disabilities and disorders of attention and longer, but not shorter, durations of anesthesia during a single exposure are associated with adverse outcomes. Over 500 preclinical studies of anesthesia neurotoxicity have demonstrated overwhelming evidence that general anesthetic drugs cause wide-spread adverse neurological effects in vitro and in immature animals, including non-human primates.

The question of how exactly early exposure to anesthesia could have an effect on brain development with long lasting consequences has remained largely unanswered. The presently disclosed subject matter, in part, addressed this question and discloses a novel mechanism whereby early postnatal exposure to anesthesia can mediate long term cognitive impairment in adults. It has previously been shown that clinically relevant concentrations of inhalational anesthetics inhibit the PDZ domain-mediated protein-protein interaction between PSD-95 or PSD-93 and NMDA receptors or nNOS. As provided hereinabove in Example 1, it was demonstrated that exposure to a single dose of isoflurane or Tat-PSD-95 PDZ2 peptide results in a loss of immature dendritic spines, impaired LTP, and cognitive abnormalities in weanling mice and cognitive impairment could be prevented by introduction of a nitric oxide (NO) donor (see also, Schaefer et al., 2019).

The presently disclosed subject matter investigates longer recovery periods to address long term/persistent effects in adult mice. In this study, it was found that the effect of a single dose of isoflurane or Tat-PSD-95 PDZ2 peptide on the developing brain results in a loss of mature ‘mushroom’ spines and causes in impairment in hippocampal dependent learning and memory in adult mice. It is further shown that the loss of mushroom spines can be prevented by a NO donor. Accordingly, these findings represent a significant advance in the field.

More particularly, the presently disclosed subject matter investigates the long-term (e.g., 5-7 weeks after exposure) effects of isoflurane (ISO) and disrupting PDZ interactions on the density of mature dendritic spines, long term potentiation (LTP), and cognition in adult mice and demonstrates that postnatal exposure to anesthesia negatively affects brain development.

In this study, postnatal day (PND) 7 mice were exposed to L5% isoflurane for 4 hrs or injected with 8 mg/kg active PSD-95 PDZ2WT peptide and/or 4 mg/kg molsidomine. Hippocampal mushroom spine density, LTP, and cognition functions were evaluated in adult mice (n=4-30). It was found that exposure of PND7 mice to ISO or PSD-95 PDZ2WT peptide causes: (1) a long term decrease in mushroom spines at PND49; (2) deficits in object recognition at PND42; (3) deficits in Y-maze at PND42 in females but not males; and (4) deficits in fear memory at PND56. The impairment in LTP at PND21 previously reported has fully recovered here at PND49. The decrease in mushroom spines was preventable by introduction of a nitric oxide (NO) donor.

This results indicate that early disruption of PDZ domain-mediated protein-protein interactions mimics isoflurane in decreasing mushroom spine density and causing memory deficits in adult mice. These results support a role for PDZ, interactions in early anesthetic exposure-produced long ter in cognitive impairment. Prevention of the decrease in mushroom spine density with a NO donor supports a role for the N-methyl-D aspartate (NMDA) receptor/PSD-95 PDZ2/neuronal nitric oxide synthase (nNOS) pathway in mediating this isoflurane-induced cellular change associated with cognitive impairment.

2.2 Background

In humans, multiple early exposures to procedures requiring anesthesia is a significant risk factor for development of learning disabilities and disorders of attention, Flick et al., 2011; Wilder et al., 2009; Hu et al., 2017; Ing and Brambrink, 2019; and Spring et al., 2012, and longer, but not shorter, durations of anesthesia during a single exposure are associated with adverse outcomes. Ing et al., 2017; Sun et al., 2016; Warner et al., 2018; Warner et al., 2019; Davidson et al., 2016; and McCann et al., 2019. Over 500 preclinical studies of anesthesia neurotoxicity have demonstrated overwhelming evidence that general anesthetic drugs cause wide-spread adverse neurological effects in vitro and in immature animals, including nonhuman primates. Jevtovic-Todorovic, 2018; Disma et al., 2018; Lin et al., 2017. Recently a group of experts identified a central goal of continuing to pursue research efforts to better understand the biological pathways underlying anesthesia neurotoxicity and to try to causally link structural changes with long-term cognitive abnormalities. Disma et al., 2018. In line with this goal, a specific molecular pathway underlying anesthesia neurotoxicity and link its disruption to a loss of mature dendritic spines and long-term cognitive abnormalities was investigated in adult mice. Our laboratory previously showed that anesthetics can disrupt PDZ domain-mediated protein-protein interactions in vitro and in vivo. Fang et al., 2003; Tao et al., 2015.

Using clinically relevant concentrations of inhalational anesthetics, PDZ domain-mediated protein interactions between PSD-95 or PSD-93 and the NMDA receptor or nNOS were dose-dependently and specifically inhibited. Fang et al., 2003. This action of anesthesia was mimicked with PSD-95 PDZ2WT peptide, which disrupts PSD-PDZ2-mediated protein interactions by binding to interaction partners. Specifically, disruption of protein-protein interactions between NMDA receptor NR2A/B subunits and PSD-95 was demonstrated. Tao and Johns, 2008. This disruption significantly reduced MAC and righting reflex EC50 indicating that this domain and protein are important for anesthetic action.

Given that: (1) the PDZ domain is a molecular target for inhalational anesthetics, Fang et al., 2003; Tao et al., 2015; (2) PSD-95 PDZ2 interacts with NMDA receptor and promotes synaptogenesis, Nikonenko et al., 2008; Kornau et al, 1995; (3) multi-innervated spine formation is prevented by deletion of the PSD-95 PDZ2 domain, Nikonenko et al, 2008; (4) disruption of PSD-PDZ2 mediated protein interactions increases anesthetic sensitivity in adult mice, Tao and Johns, 2008; and (5) neonatal disruption of PSD-PDZ2 mediated protein interactions leads to a decrease of long thin spines, impairs LTP, and impairs novel object recognition in weanling mice, Schaefer et al., 2019, it was thought that early postnatal disruption of PDZ domain mediated protein-protein interactions can have persistent effects into adulthood including long term loss of mature spines and impaired cognitive functioning.

The presently disclosed subject matter, in part, investigates the long term effect of disrupting PSD-95 PDZ2 domain-mediated protein-protein interactions on mature dendritic spines, plasticity, and cognition. The long term outcome in adult mice of disrupting PSD-95 PDZ2 domain-mediated protein-protein interactions early in development on dendritic spine maturation, long term potentiation, and hippocampal dependent behaviors including novel object recognition memory (non-spatial), Y-maze reference memory (spatial), and contextual fear memory was examined in vivo. To specifically determine the involvement of the NMDAR NR2-PSD-95 PDZ2-nNOS pathway an NO donor was introduced at the time of isoflurane anesthesia or PSD-95 PDZ2 exposure to test if spine loss is preventable.

2.3.1 Materials and Methods

This study was carried out with approval from the Animal Care and Use Committee at Johns Hopkins University and was consistent with the National Institutes of Health Guide for the Care and Use of Laboratory Animals. No surgery was performed, and all efforts were made to minimize animal suffering and reduce the number of animals used. C57BL6 wild type (WT) male and female mice were used in our study. On PND7, animals from each litter were randomly assigned to control and treatment groups. Mice were maintained under standard lab housing with 12 h light/dark cycle. Water and food were available ad libitum until mice were transported to the laboratory approximately 1 h before the experiments.

2.3.2 Anesthesia, Peptide, and Molsidomine Injections

PND7 control and experimental mice were placed in a clear plastic cone and body temperature maintained by a heating blanket set to 35° C. Heart rate (HR) and oxyhemoglobin saturation (SpO₂) were continuously monitored using pulse-oximetry (PhysioSuite, Kent Scientific). Arterial blood was collected by terminal cardiac puncture of the left ventricle from sentinels and pH (pHa) determined using a blood gas machine (ABL800 FLEX Series Radiometer). At the end of the 4-hour exposure to isoflurane (1.5% in 100% 02), our data (mean±SD) suggested mice were adequately oxygenated (SpO₂, 93±6, n=5) and were within acceptable ranges for HR (611±84, n=5) and pHa (pH, 7.26±0.05, n=2). Naïve control animals were left with the dams. Control ‘CON’ animals were exposed to oxygen only. Anesthesia was initiated with 2.4% isoflurane in oxygen for 2 min and tapered down to 1.5% within 15 min. Exposure to 1.5% isoflurane was continued for 3 hours and 45 min (total 4 hours ISO). At the end of the exposure animals were maintained in oxygen on the heating blanket for 10 min then returned to dams. The purified fusion peptides, active Tat-PSD-95 PDZ2WT (referred to as PDZ2WT in manuscript) or inactive Tat-PSD-95 PDZ2MUT (referred to as PDZ2MUT in manuscript), at 8 mg/kg were injected into mice intraperitoneally (ip) in 150 μL of PBS and 10% glycerol, as previously described. Tao and Johns, 2008. Purification of fusion peptides was performed by Creative BioMart (Shirley, N.Y.) and verified by Coomassie blue staining and Western blot analysis and then stored in 10% glycerol/phosphate-buffered saline at −80° C. until use. The Tat-PSD-95 PDZ2WT and MUT plasmids used to generate proteins containing an amino-terminal, in-frame, 11-amino-acid, minimal transduction domain (residues 47-57 of human immunodeficiency virus Tat protein) termed Tat. Inactive control plasmid, mutated Tat-PSD-95 PDZ2, has three sites critical for interactions between NMDARs and PSD-95 mutated (K165T, L170R. and H182L). Fang et al., 2003. The NO donor Molsidomine [(N-[ethoxycarbonyl]-3-[4-morpholinosydnomine] (Sigma, St. Louis, Mo.) was injected at 4 mg/kg into mice ip in 100 μL sterile saline as previously described. Control animals were injected ip with the vehicle (saline).

2.3.3 Golgi Staining, Microscopy, and Spine Reconstruction

PND49 mice were deeply anesthetized and perfused transcardially with a brief flush of 0.01 M phosphate-buffered saline (pH 7.4) followed by 50 mL of 4% paraformaldehyde in 0.1 M phosphate buffer (pH 7.4). After the perfusion, the brains were removed and hippocampi were grossly dissected and stained using FD Rapid GolgiStain™ Kit, (FD NeuroTechnologies, Inc., Columbia, Md.) as per vendors instructions. Briefly, tissue was immersed in AB impregnation solution at room temperature in the dark for 2 weeks. Impregnation solution was replaced after the first overnight on the next day. Tissue was transferred to solution C for 72 hours. Hippocampi were embedded in tissue freezing medium (TFM) and stored at −80° C. 60 μm sections were cut on a cryostat at −20° C., mounted onto gelatin coated slides, and air dried overnight. Slides were rinsed in Milli-Q water, developed in the working solution DE for 10 min, rinsed, dehydrated in EtOH, cleared in xylene, and mounted with Permount®.

Two different imaging fields per mouse each containing at least three unique dendritic segments (6 segments total per mouse), which contain dorsal hippocampus were imaged. The 6 segments were averaged per mouse to contribute one datapoint per mouse. Dentate granule cells (DGCs) were identified by their location within the dentate gyrus (DG) and their distinct morphology. Spines along secondary and tertiary dendrites of these neurons were selected for analysis. Schaefer et al., 2019. Z-stacks of Golgi stained dendrites (optical section thickness=0.3 μm, i.e. 50-100 images per stack) were taken at 630× magnification on a Leica SPE confocal microscope. Spine analysis was performed as described in Risher et al., 2014, using the freely available RECONSTRUCT software. Fiala, 2005.

2.3.4 Electrophysiology

2.3.4.1 Slice preparation. Six weeks after exposure (PND49-52) mice were euthanized and coronal brain slices containing central part of hippocampus (300-μm thick) were made from Leica VT 1200S vibrotome in ice-cold ACSF containing (in mM): 128 NaCl, 3 KCl, 26 NaHCO₃, 1 NaH₂PO₄, 1 MgSO₄, 10 glucose and 2 CaCl₂) and saturated with 95% 02 and 5% CO₂. The slices were incubated for at least 1 hr at room temperature (22-24° C.) in the interface-type holding chamber filled with ACSF. Then a slice was transferred to the recording chamber, where AC SF was perfused at a rate of 1.5-2.0 mL/min at room temperature. 2.3.4.2 Extracellular field-potential recordings. Synaptic responses were recorded using a MultiClamp 700B amplifier, and the signal was digitized with Digidita. 1440A, analyzed with pClamp10 and stored on a personal computer. Extracellular recordings of field excitatory postsynaptic potentials (fEPSPs) were made from the stratum radiatum of the hippocampal CA1 area. Evoked responses were elicited with 0.1-msec constant-current pulses through a concentric electrode in the Schaffer collateral pathway every 30 sec at an intensity sufficient to elicit 40-50% maximal EPSPs. After establishing a stable baseline for 20 min, LTP was induced by applying three trains of 100 Hz×1 sec high frequency stimulus (HFS) 20 sec apart at the baseline stimulus intensity. Measurements of the fEPSP slopes were made during the rising phase (5-50% of the peak) and the values normalized to the mean values recorded in 20-min baseline. The median of normalized fEPSP slopes 55-60 min after HFS was used for comparison between groups. Each animal contributed one data point to obtain the median for each group.

2.3.5 Novel Object Recognition

The novel object recognition (NOR) procedure was based upon the original procedure, Ennaceur and Delacour, 1988, and was used to assess nonspatial hippocampal memory. Clark et al., 2000; Baker and Kim, 2002; and Reger et al., 2009. It consisted of a training ‘familiarization’ phase followed by a testing phase. During training, mice were allowed to freely explore within an opaque box (40 cm W×40 cm L×34 cm H) containing two identical objects for 10 min. Data were recorded with video camera and time spent with each object was recorded using ANYmaze software (Stoelting). Object investigation time was determined by the amount of time the mouse spent in the zone immediately surrounding the object. Only mice that investigated the objects for at least 10 sec (criterion) were taken forward to the testing phase (n=5 animals were not taken forward as they did not meet criterion). After 2 h, object recognition was tested, using the same procedure as in training except that a novel object was substituted for one of the familiar training objects and mice were allowed to explore for 5 min. Mice inherently prefer to explore novel objects; thus, a preference for the novel object indicates intact memory for the familiar object.

2.3.6 Y-Maze

The Y-maze test was as described. Kang et al., 2017. In the Y-maze test, mice were released from the start arm (no visual cue) and allowed to habituate to only 1 out of 2 possible choice arms (overt visual cue) for 15 minutes. This was followed at 24 hours later by the recognition phase in which the animal could choose between the 2 choice arms after being released from the start arm. The timed trials (5 minutes) were video recorded for total exploration time in each choice arm.

2.3.7 Fear Behavior Testing

2.3.7.1 Contextual fear conditioning. Six weeks after anesthetic exposure, the PND49 animals were acclimated to the behavior testing room for 60 min. The conditioning trial protocol was set up to allow testing of both cued and contextual fear behavior, although only contextual fear testing was performed. The conditioning trial consisted of a 3-min exploration period in conditioning chambers (Coulbourn Instruments, Whitehall, Pa.) followed by three conditioned stimulus (CS)-unconditioned stimulus (US) pairings separated by 1.0 min each: US, 0.5 mA foot shock intensity, 1 s duration; CS, 90 db white noise tone, 30 s duration. The US was delivered during the last second of the CS presentation.

2.3.7.2 Contextual fear testing. At 1 week after the conditioning, the PND56 mice were acclimated to the behavior room for 60 min. Then, they were placed into the chambers for 7 min and percent of freezing duration was captured by FreezeScan camera software (Clever Sys., Reston, Va.). The average percent freezing duration was calculated for the first 2.5 min of the testing trial.

2.3.8 Statistical Analysis

Statistical analysis was carried out by unpaired, two-tailed, Mann-Whitney tests with Graphpad Prism version 7.0 software (Graphpad Inc., La Jolla, Calif.). Mann-Whitney, two-tailed test was used to compare CON versus ISO and PDZ2MUT versus PDZ2WT groups (spine analysis with and without NO donor, NOR, Y-maze, fear memory, and LTP). Data are expressed as median, interquartile range respectively, and statistical significance was set at P<0.05. *p<0.05, **p<0.01, ***p<0.001, and ****p<0.0001. Sample sizes were chosen based on previous experience and/or published literature. A sample size of N>4 was chosen for spine analysis because an N=4 was sufficient to show differences in hippocampal proportional densities of different types of spines following excitoxicity. Gonzalez-Burgos et al., 2009. A sample size of N>4 was chosen for LTP measurements because an N=3 was sufficient to show differences in LTP amplitude induced by NMDA antagonist. Pigott and Garthwaite, 2016. A sample size of N=5 to 25 was chosen for NOR because sample sizes of 10 were sufficient to show age specific differences in rodents. Reger et al., 2009; Benice et al., 2006. A sample size of N>7 was chosen for Y-maze because sample sizes of 7 were sufficient to show specific differences in mice in a similar Y-maze test. Shirai et al., 2010. In all experiments each animal contributed one data point to obtain the median for each group. 2.4 Results

2.4.1 Neonatal exposure to isoflurane or PDZ2WT peptide leads to a decrease in hippocampal dendritic mushroom spines in adult mice. To determine whether inhaled anesthetics interfere with spinogenesis and have long lasting effects by disrupting synaptic PDZ interactions in the developing hippocampus, the impact of isoflurane and disrupting PSD-95 PDZ2 domain-mediated protein-protein interactions on mature dendritic spines in male and female PND49 mice was investigated six weeks following an exposure at PND7. Neonatal WT mouse pups (PND7) were exposed to isoflurane or O₂ for 4 hrs. A separate cohort of WT animals were also exposed to PDZ2MUT or PDZ2WT peptides. Animals were harvested six weeks later (PND49) for rapid Golgi staining to visualize hippocampal dendritic spines within the superior blade of the DG. Mushroom spines were quantified along the dendritic segments distal to the first and second branch points. Data were analyzed using Mann Whitney tests.

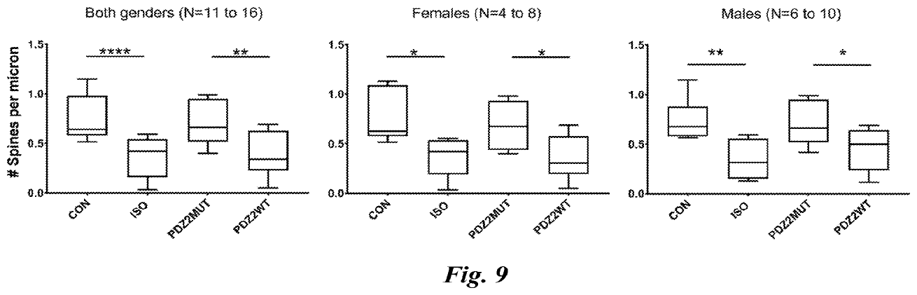

Isoflurane or PDZ2WT peptide had a significant effect on number of mushroom type protrusions present at 7-weeks of age (width>0.6 μm; FIG. 9, median, IQ: Left plot, both genders mixed: CON (0.64, 0.58 to 0.98), ISO (0.42, 0.16 to 0.54), p<0.0001; PDZ2MUT (0.64, 0.51 to 0.95) vs PDZ2WT (0.34, 0.23 to 0.63), p=004). Middle plot, females: CON (0.63, 0.58 to 1.09), ISO (0.42, 0.19 to 0.54), p=0.017; PDZ2 MIT (0.68, 0.44 to 0.93) vs PDZ2WT (0.30, 0.20 to 0.58), p=0.048). Right plot, males: CON (0.68, 0.58 to 0.88), ISO (0.32, 0.15 to 0.55), p=0.003; PDZ2MUT (0.66, 0.52 to 0.95) vs PDZ2WT (0.50, 0.24 to 0.64), p=0.043).

2.4.2 Neonatal exposure to isoflurane or PDZ2WT peptide impairs object recognition memory in adult mice. To determine whether the disruption of synaptic PDZ interactions contributes to cognitive impairment after early anesthetic exposure and has long lasting effects, the impact of isoflurane and disrupting PSD-95 PDZ2 domain-mediated protein-protein interactions on non-spatial memory was investigated by assessing hippocampal dependent object recognition in PND42 mice five weeks after exposure at PND7.

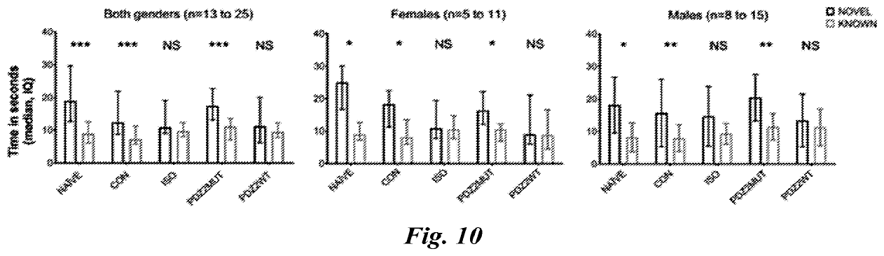

All control mice (Naïve, CON, and PDZ2MUT) were able to discriminate between novel and known objects revealed by significantly increased amounts of time investigating the novel object over the known object (FIG. 10; median, IQ for novel vs known: mixed gender NAÏVE (19, 13 to 30 vs 9, 6 to 13), p=0.0002; mixed gender CON (12, 9 to 22 vs 7, 6 to 12), p=0.0002; mixed gender PDZ2MUT (19, 13 to 23 vs 12, 7 to 14), p=0.0010 two-tailed Mann-Whitney test novel vs. known. Female NAÏVE (25, 17 to 30 vs 9, 7 to 13), p=0.0159; female CON (18, 11 to 22 vs 8, 6 to 14), p=0.0147; female PDZ2MUT (16, 12 to 22 vs 11, 7 to 12), p=0.0158. Male NAÏVE (14, 12 to 28 vs 8, 4 to 13), p=0.0104; male CON (11, 9 to 21 vs 6, 6 to 10), p=0.0048; male PDZ2MUT (19, 15 to 26 vs 12, 8 to 15), p=0.0061). In contrast, experimental mice (ISO and PDZ2WT) showed no significant increase in investigation time between the novel and known objects (FIG. 10:mixed gender ISO (11, 9 to 19 vs 10, 8 to 12), p=0.06; mixed gender PDZ2WT (11, 6 to 20 vs 10, 8 to 12), p=0.38. Female ISO (11, 8 to 1.9 vs 11, 8 to 15), p=0.6842; female PDZ2WT (9, 6 to 21 vs 9, 5 to 17), p=0.7959. Male ISO (11, 9 to 19 vs 9, 8 to 11), p=0.1273; male PDZ2WT (12, 7 to 21 vs 11, 8 to 12), p=0.5613.

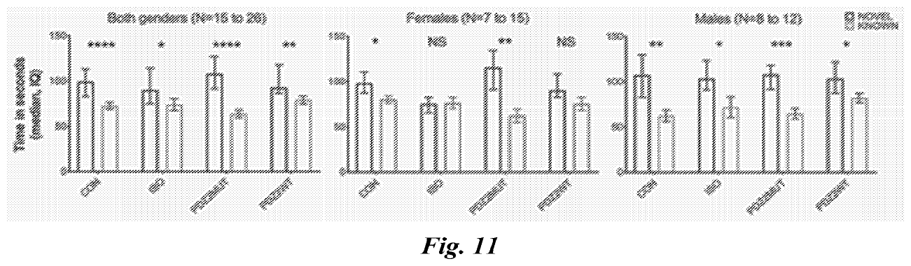

2.4.3 Neonatal exposure to isoflurane or PDZ2WT peptide influences performance of adult mice in Y-maze spatial recognition memory test with females exhibiting poorer performance. To deter mine whether the disruption of synaptic PDZ, interactions contributes to cognitive impairment after early anesthetic exposure and has long lasting effects, the impact of isoflurane and disrupting PSD-95 PDZ2 domain-mediated protein-protein interactions on spatial reference memory was investigated by assessing arm recognition in PND42 mice five weeks after exposure at PND7. Control mice (CON and PDZ2MUT) were able to discriminate between novel and known anus revealed by significantly increased amounts of time investigating the novel arm over the known arm (FIG. 11; median, IQ for novel vs known: mixed gender CON (99, 83 to 113 vs 72, 61 to 86), p<0.0001; mixed gender PDZ2MUT (108, 92 to 127 vs 63, 56 to 70), p<0.0001 two-tailed Mann-Whitney test novel vs. known. Female CON (98, 88 to 111 vs 79, 66 to 87), p=0.0115; female PDZ2MUT (116, 91 to 135 vs 63, 60 to 78), p=0.0011. Male CON (107, 83 to 130 vs 59, 48 to 72), p=0.0019; male PDZ2MUT (108, 92 to 118 vs 62, 56 to 70), p=0.0001). In contrast, female experimental mice (ISO and PDZ2WT) showed no significant increase in investigation time between the novel and known arms (FIG. 11: female ISO (83, 65 to 130 vs 72, 67 to 96), p=0.3829; female PDZ2WT (90, 83 to 108 vs 67, 57 to 96), p=0.1359. The mixed gender and male groups showed less of a significant difference (magnitude of difference in investigation time between novel and known arm) in experimental groups compared to controls. Mixed gender ISO (102, 82 to 126 vs 73, 52 to 97), p=0.0145; mixed gender PDZ2WT (93, 86 to 118 vs 81, 64 to 96), p=0.0035. Male ISO (103, 91 to 123 vs 78, 44 to 100), p=0.0379; male PDZ2WT (104, 87 to 122 vs 81, 75 to 96), p=0.0122.

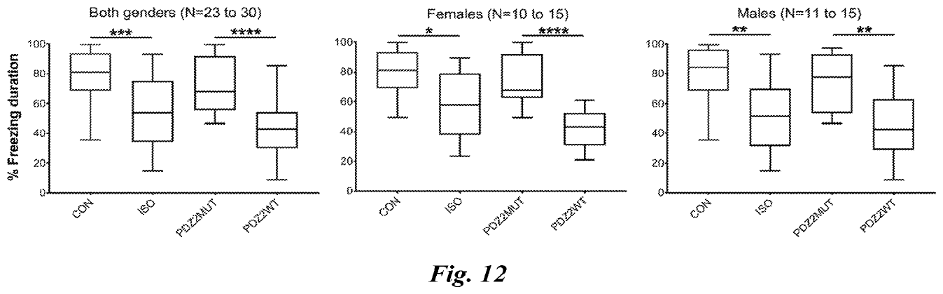

2.4.4 Neonatal exposure to isoflurane or PDZ2WT peptide leads to contextual fear memory impairment in PND56 adult mice. Contextual fear learning is a form of Pavlovian conditioning elicited by pairing a neutral conditioned stimulus (CS; for example, sound or context) with an aversive unconditioned stimulus (US). Acquisition of a context-US association usually requires both the hippocampus and amygdala. Daumas et al., 2005; Phillips and LeDous, 1992. Whether early exposure to isoflurane or disruption of PSD-95 PDZ2 domain-mediated protein-protein interactions impairs the ability of mice to remotely retrieve information about the stored association (memory) was tested. Contextual fear testing was performed in PND56 mice 1 week after conditioning and 7 weeks after exposure (FIG. 12). Isoflurane and PDZ2WT exposed mice exhibited significantly reduced freezing behavior compared to controls. FIG. 12. median, IQ: Left plot, mixed genders: CON (81, 69 to 93) vs ISO (58, 38 to 79), p<0.0161, PDZ2MUT (68, 63 to 92) vs PDZ2WT (43, 31 to 53), p<0001. Middle plot, females: CON (81, 69 to 93) vs ISO (58, 38 to 79), p=0.0161; PDZ2MUT (68, 63 to 92) vs PDZ2WT (43, 31 to 53), p<0.0001. Right plot, males: CON (84, 69 to 96) vs ISO (52, 31 to 70), p=0.003, PDZ2MUT (78, 54 to 93) vs PDZ2WT (43, 29 to 63), p=0.003.

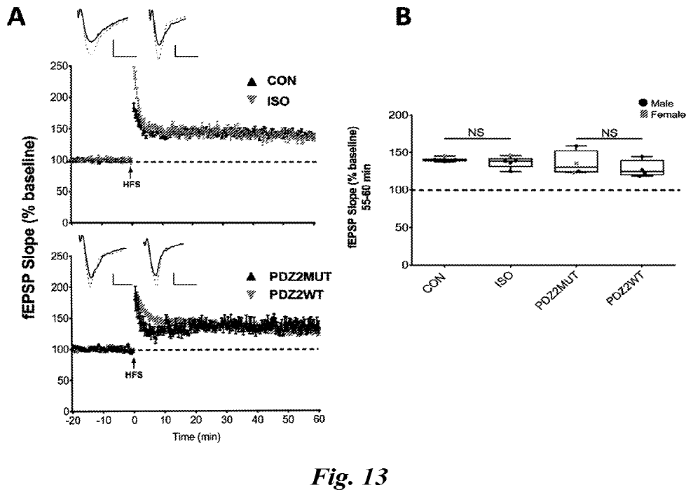

2.4.5 Neonatal exposure to isoflurane or PDZ2WT peptide does not result in impaired LTP in adult PND49 mice. Previously, it was shown that early PND7 exposure to isoflurane or disruption of PSD-95 PDZ2 domain-mediated protein-protein interactions impaired LTP in hippocampal slices prepared from mice at PND21 (two weeks after exposure). Here, the electrophysiological effects following an even longer recovery period following PND7 isoflurane or PDZ2WT peptide exposure was assessed in hippocampal slices prepared from mice at PND49. Six weeks after exposure, robust LTP can be induced in all mice (FIG. 13A). No significant differences were observed between control and experimental groups. FIG. 13B; median, IQ:CON (140, 138 to 142 vs ISO 139, 131 to 142), p=0.4286 or inactive PDZ2MUT peptide (130, 124 to 153 vs active peptide PDZ2WT 125, 120 to 140), p=0.4857 55-60 min after IFS.

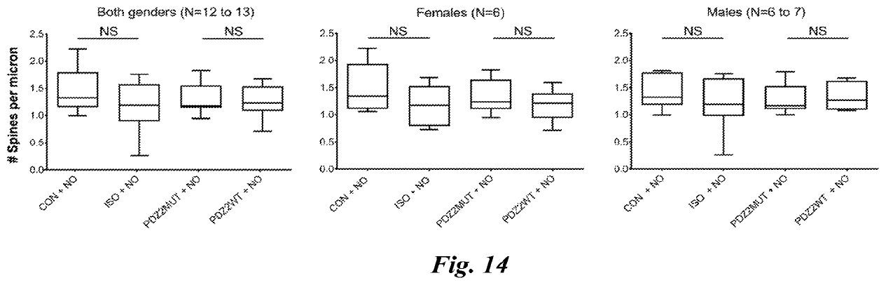

2.4.6 Treatment with NO donor prevents isoflurane or PDZ2WT induced decrease in hippocampal dendritic mushroom spines in adult PND49 mice. Treatment with NO donor prevents the decrease in mushroom spines caused by isoflurane or PDZ2WT peptide. There is no longer a significant difference between isoflurane or PDZ2WT and their respective controls when NO donor is added (FIG. 14; median, IQ: mixed gender CON+NO (1.33, 1.16 to 1.80) vs ISO+NO (1.19, 0.90 to 1.58), p=0.0678; mixed gender PDZ2MUT+NO (1.18, 1.15 to 1.56) vs PDZ22WT+NO (1.23, 1.08 to 1.54), p=0.6297. Female CON+NO (1.34, 1.11 to 1.94) vs ISO+NO (1.18, 0.79 to 1.53), p=0.3095, Female PDZ2MUT+NO (1.24, 1.11 to 1.65) vs PDZ22WT+NO (1.21, 0.94 to 1.40), p=0.4848. Male CON+NO (1.33, to 1.18 to 1.78) vs ISO+NO (1.19, 0.98 to 1.68), p=0.1807; Male PDZ2MUT+NO (1.17, 1.11 to 1.53) vs PDZ22WT+NO (1.27, 1.09 to 1.63), p>0.9999. Data were analyzed with Mann-Whitney test.

2.5 Discussion

Millions of children in the USA are exposed to general anesthetic agents each year, with millions more exposed around the world. Ing and Brambrink, 2019; Rabbitts et al., 2010; and Tzong et al., 2012. Results from clinical studies have been mixed with some studies showing an association between anesthetic exposure and neurodevelopmental deficit and others not. Recent results from three high qualified major clinical studies (Mayo Anesthesia Safety in Kids (MASK), Pediatric Anesthesia Neurodevelopment Assessment (PANDA), and General Anesthesia Spinal (GAS)) indicate no effect on cognitive performance, IQ, and operant test battery (OTB) in children after short exposure to anesthesia during surgery (median durations 45, 80, 54 min respectively) in early infancy. Sun et al., 2016; Warner et al., 2018; Warner et al., 2019; Davidson et al., 2016, McCann et al., 2019, and Zaccariello et al., 2019. From these findings most experts would agree that single, brief anesthesia exposure in early infancy does not result in significant neurodevelopmental defects in healthy individuals.37 Shao et al., 2019. Nevertheless, these studies still leave important issues unsolved including whether neurodevelopmental defects might exist in untested neurocognitive domains or are associated with vulnerable populations and longer or multiple anesthesia exposures. Continued concern and current evidence that lengthy (>3 hours) or repeated exposure to anesthetic agents in humans during early postnatal life may cause long-term neurodevelopmental defects most certainly warrants further research. Hu et al., 2017; Ing and Brambrink, 2019; Ing et al., 2017; Warner et al., 2018; Communication FDS, 2017; Communication FDS, 2016.

To better understand the mechanisms underlying anesthesia neurotoxicity and to try to link structural changes with long-term cognitive abnormalities, one specific molecular target of anesthesia was investigated. It was previously reported that inhalational anesthetics, including halothane, isoflurane, and sevoflurane, can disrupt PDZ domain-mediated protein-protein interactions in vitro and in vivo, Fang et al., 2003, Tao et al., 2015, and specifically inhibit the PDZ domain-mediated protein interaction between PSD-95 or PSD-93 and the NMDA receptor NR2 subunits or nNOS. Fang et al., 2003; Tao and Johns, 2008.

Recently, the effects of this disruption, in vivo, in the context of isoflurane exposure was determined by specifically mimicking this one action of anesthesia with PDZ2WT peptide. Schaefer et al., 2019. PDZ2WT peptide disrupts PSD-PDZ2-mediated protein interactions by binding to interaction partners such as NMDA receptor NR2 (see FIG. 1 in Schaefer et al., 2019; Tao and Johns, 2008). It was found that isoflurane and disruption of PSD-95 PDZ2 domain-mediated protein-protein interactions at PND7 alters spine morphology, impairs LTP, and impairs memory in weanling aged mice (i.e., PND21). In addition, introduction of NO donor at the time of exposure prevented impairment in LTP and recognition memory further implicating the involvement of the NMDAR NR2-PSD-95 PDZ2-nNOS signaling pathway in early anesthetic exposure-produced cognitive impairment. One aspect of the presently disclosed subject matter was to determine what changes occur following our exposure paradigm in mice allowed to recover for longer periods of time (i.e., adulthood). Anesthesia exposure, nNOS activity, and modulation of membrane-associated guanvlate kinase levels have all been associated with spine morphological and density changes. Nikonenko et al., 2008; Vickers et al., 2006; Nidonenko et al., 2002. Dendritic spines are critical for learning and memory functions. Grienberger et al., 2015.

Whether neonatal anesthetic exposure could lead to a persistent change in mature spines of hippocampal dentate granule cells was addressed. Mushroom spines typically represent long-lasting, stable synaptic connections. Bourne and Harris, 2007. Mushroom spines were measured on secondary and tertiary dendrites which receive input from medial entorhinal cortex in the middle molecular layer of the hippocampus. This axis has been indicated to be involved in spatial memory. Hafting et al., 2005. Results of our spine analysis indicate a significant loss of mushroom spines (width>0.6 microns) in isoflurane or PDZ2WT exposed animals compared to controls in both males and females measured six weeks after exposure. Others have also shown selective loss of spines from anesthesia such as the persistent decrease (up to 90 days) in spine density of spines with head diameter between 0.3-0.4 microns, Briner et al., 2011, and mushroom spines at PND60. Kang et al., 2017. The reduction in mushroom spine number suggests a substantial loss of synapses and might involve a role in circuitry development and permanently altered neural connectivity. A decrease in mushroom spines could reasonably account for reduced cognitive performance reported in individuals with early postnatal exposure to anesthesia. Whether the disruption of synaptic PDZ interactions contributes to persistent learning and memory deficits after early anesthetic exposure was explored. Cognitive performance in adult mice was assessed using three different behavior tests that have previously indicated impairment following early exposure to anesthesia and that involve different types of hippocampal dependent learning and memory including non-spatial recognition memory (NOR), Schaefer et al., 2019; Zhu et al., 2010; Shih et al, 2012, spatial reference memory (Y-maze), Kang et al., 2017; Kraeuter et al., 2019, and contextual fear memory (fear conditioning). Satomoto et al., 2009; Man et al., 2015. Previously it was found that weanling mice exposed early to isoflurane or injected with PDZ2WT peptide exhibited reduced recognition memory performance as compared with controls. Schaefer et al., 2019. Here, whether this behavior impairment persists to adulthood was investigated. It was found that adult PND42 male and female mice exposed early to isoflurane or injected with PDZ2WT peptide also exhibited reduced recognition memory performance. These results indicate that intact PSD-PDZ2-mediated protein interactions are important for hippocampal-dependent recognition memory performance in weanling and adult mice. Persistent long-term spatial reference memory was tested in adult mice via Y-maze with a 24-h inter-trial interval. It was found that isoflurane and disrupting synaptic PDZ, interactions results in a lasting reduction in performance in females in this spatial task dependent on the hippocampus and potentially sensitive to a decrease in mature spines in the middle molecular layer of the hippocampus. These results are similar to those of Gonzales et al., 2015, who demonstrated propofol-exposed females had impaired performance on the spontaneous alternation Y-maze task, suggesting possible working memory disruptions. In another study, examination of spatial reference memory revealed that female, hut not male, neonatal rats exposed to anesthesia showed slowing of acquisition rates suggesting that females were more vulnerable to anesthesia induced cognitive impairment. Boscolo et al., 2013. Long-lasting impairments in fear conditioning, persisting into adulthood, have been observed after exposure of neonatal mice to sevoflurane. Satomoto et al., 2009. Contextual fear tests evaluate hippocampus-dependent learning and memory functions. Kim and Fanselow, 1992. Long-term memory was assessed based on the freezing reaction of adult mice in response to a previously conditioned context. The freezing response to the same context in mice with neonatal exposure to isoflurane or PDZ2WT peptide was reduced significantly compared with controls after a 1-week retention delay at 8 weeks of age. Thus, it was found isoflurane or disruption of synaptic PDZ interactions causes persistent memory deficits later in adulthood as evidenced by decreased freezing response at 8 weeks of age (one week after fear conditioning and seven weeks after exposure).

To further explore whether the disruption of synaptic PDZ interactions could contribute to persistent learning and memory deficits through altered synaptic function after early anesthetic exposure, LTP in adult mice was investigated. Cooke and Bliss, 2006. LTP is widely considered as a major cellular mechanism underlying learning and memory. Cooke and Bliss, 2006. Hippocampal granule neurons, that are shown here in adults to have decreased mushroom spine density after early exposure to isoflurane or PDZ2WT peptide, emit mossy fibers that synapse on pyramidal neurons of area CA3 of Ammon's horn, which synapse on pyramidal neurons of area CA1 of Ammon's horn. These connections are involved in LTP and long-term depression. Thus, whether the long-tei in decrease in mushroom spines parallels suppressed LTP was of interest.

Previous work demonstrated early exposure to a combination anesthetic induced a profound suppression of LTP in the hippocampus of adolescent rats. Jevtovic-Todorovic et al., 2003. In addition, it was found suppression of LTP in the hippocampus of weanling mice exposed early to isoflurane and PDZ2WT peptide as compared to controls. Thus disrupting PDZ domain-mediated protein interactions mimicked the effect of isoflurane on LTP. These results suggested synaptic PDZ interactions may contribute to the mechanism underlying anesthesia induced impairment in synaptic function in mice and therefore could contribute to learning and memory deficits. Here, it was found the suppression of LTP by either isoflurane or PDZ2WT peptide had fully recovered by 7-weeks of age. Thus exposure to isoflurane or PDZ2WT peptide causes long-lasting (2-weeks after exposure), Schaefer et al., 2019, but not permanent (6-weeks after exposure), impairment of synaptic plasticity in area CA1 of the hippocampus.

It has been demonstrated that PDZ domain-mediated interactions between PSD-95 or PSD-93 and NMDA receptors or nNOS are disrupted by clinically relevant concentrations of anesthetics. Fang et al., 2003. Without wishing to be bound to any one particular theory, it was thought that introduction of certain downstream signaling components during the time of isoflurane or PDZ2WT peptide exposure may prevent the decrease in mushroom spines. Indeed, treatment with the NO donor, Molsidomine, prevents the decrease in mushroom spine density caused by isoflurane or PDZ2WT peptide suggesting the effect of NO is downstream of the disrupted PDZ interactions. These results support the involvement of the NMDAR NR2-PSD-95 PDZ2-nNOS signaling pathway in isoflurane mediated decrease in mushroom spine density. A better understanding of how NO signaling affects mushroom spine density is an important area to pursue.

In summary, our findings indicate that a single 4-hr exposure of infant mice to 1.5% isoflurane or targeted disruption of PSD-PDZ2-mediated protein interactions (i.e. a specific molecular target of isoflurane) with PDZ2WT peptide results in a persistent decrease in mushroom spine density and impairments in hippocampal dependent memory (non-spatial, spatial, and contextual) in adult mice. The observed decrease in mushroom spine density can be prevented by introduction of an NO donor suggesting the involvement of NMDAR NR2-PSD-95 PDZ2-nNOS signaling pathway in these processes.

### Example 3

### Mechanistic Actions of PDZ, Domain Mediated Protein Interactions on Neural Development and Anesthetic-Mediated Neurotoxicity

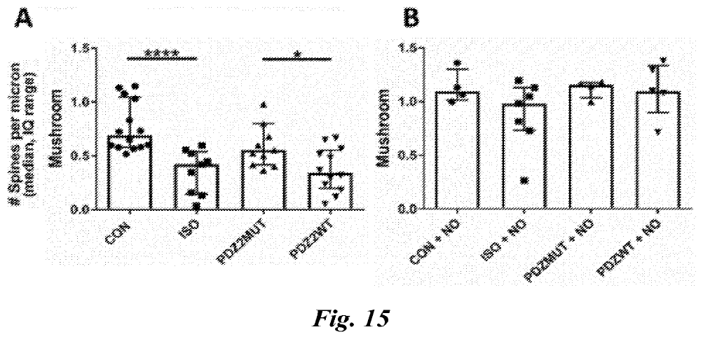

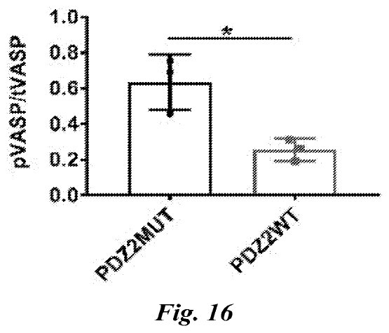

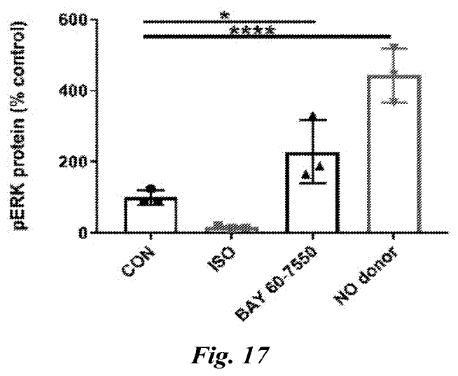

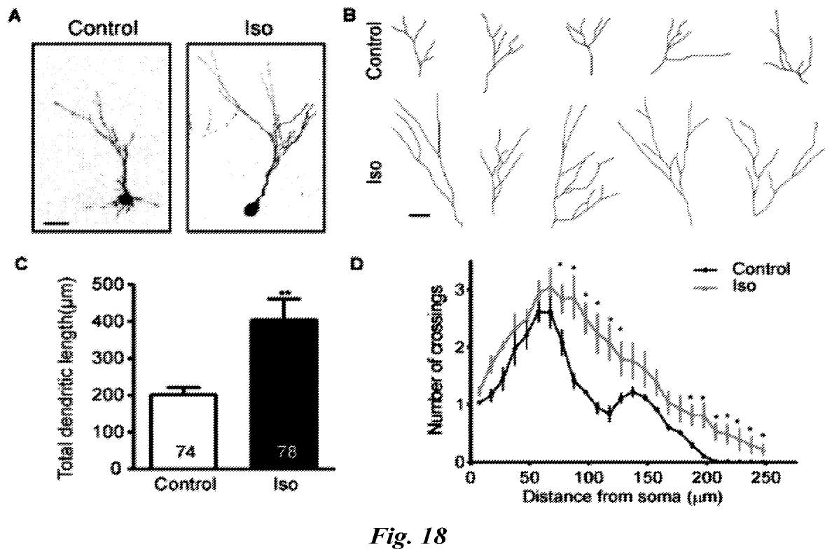

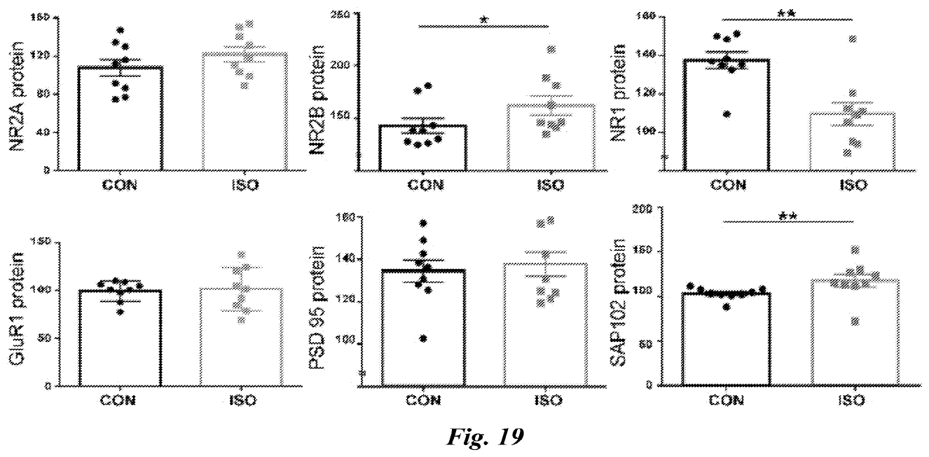

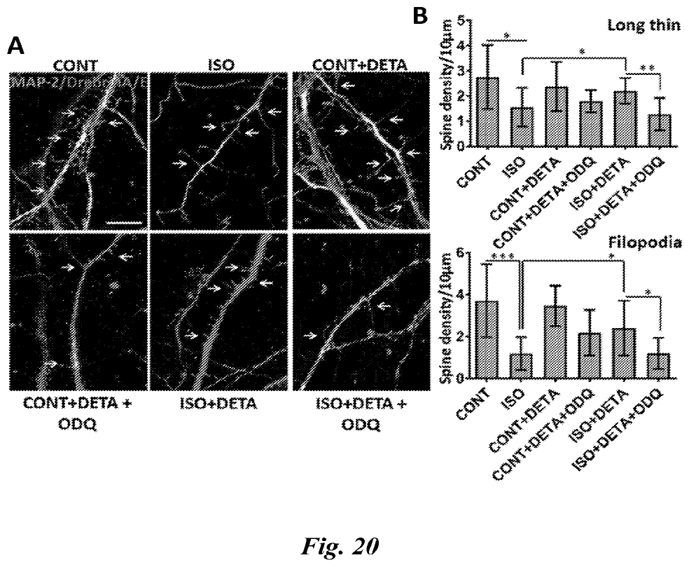

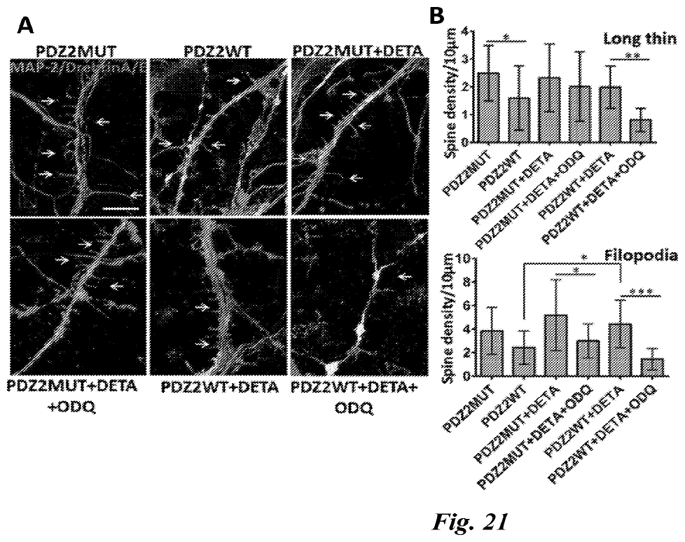

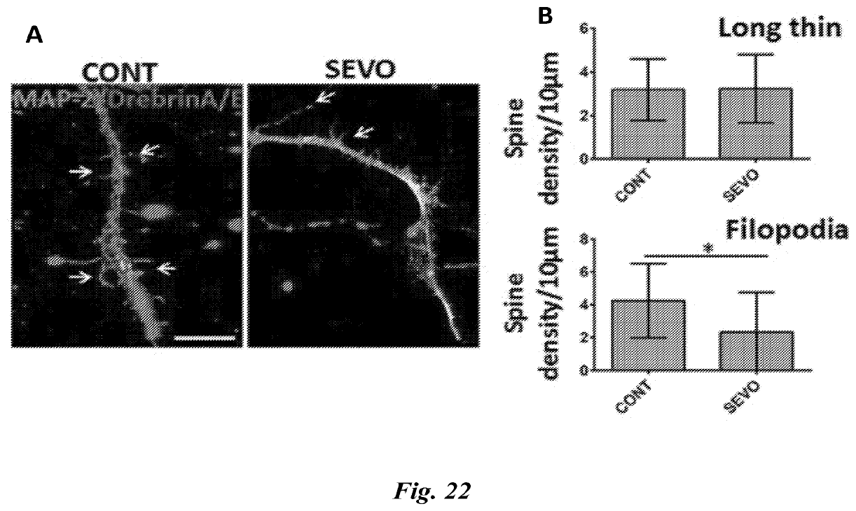

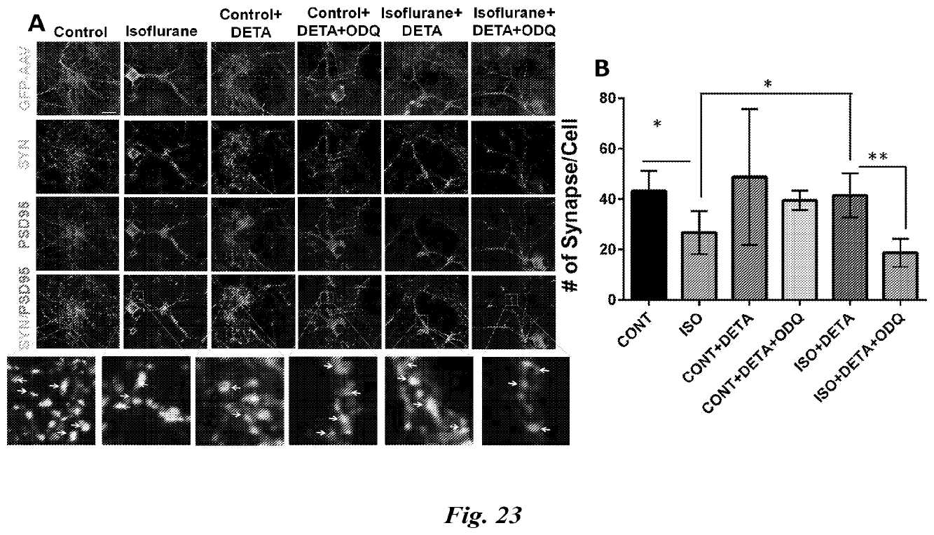

This example and the associated figures demonstrate that: (1) isoflurane and disruption of PSD95-PDZ2 domain interactions during early development leads to a loss of immature spines in weanlings, Schaefer et al., 2019, (see FIG. 3A-3C hereinabove) and a loss of mature spines in adults (FIG. 15); (2) nitric oxide (NO) donor prevents the loss of mushroom spines (FIG. 15); (3) neonatal isoflurane and disruption of PSD95-PDZ2 domain interactions impairs long term potentiation, Schaefer et al., 2019, (see FIG. 5 and FIG. 7 described hereinabove) and memory, Schaefer et al., 2019, (see FIG. 6 and FIG. 8 hereinabove) and can be prevented by introduction of NO donor (see FIG. 5, FIG. 7, FIG. 6, and FIG. 8 described hereinabove); (4) neonatal disruption of PSD95-PDZ2 domain interactions results in decreased vasodilator-stimulated phosphoprotein (VASP) phosphorylation supporting our hypothesis that isoflurane causes downregulation of NO-cyclic guanosine monophosphate (cGMP)-protein kinase G (PKG)-pVASP signaling (FIG. 16); (5) NO donor and phosphodiesterase (PDE) inhibitor increase extracellular signal-regulated kinase (ERK) phosphorylation supporting our hypothesis that regulation of NO-cGMP-PKG alters ERK signaling (FIG. 17); (6) postnatal exposure to isoflurane causes an increase in dendritic arbor length, Kang et al., 2017, (FIG. 18) and a prolonged increase in expression of synaptic NMDA receptor (NR) 2B and synapse-associated protein (SAP)102 (FIG. 19) supporting our hypothesis that isoflurane delays displacement of SAP102/NR2B-NMDAR complexes; (7) in vitro results, which are independent of animal physiology, corroborate in vivo results (FIGS. 20-22); and (8) isoflurane exposure causes loss of synapses at DIV14, which can be prevented with NO donor and this prevention is attenuated with sGC inhibitor (FIG. 23).

Without wishing to be bound to any one particular theory, it is thought that that early exposure to anesthesia alters neural development by disrupting PDZ-domain mediated interactions causing uncoupling of PSD-95-NMDAR associated synaptic complexes resulting in inhibition of prominent downstream signaling pathways critical to dendritic spine, synapse, and arbor development.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Jackson W M, Gray C D, Jiang D, Schaefer M L, Connor C, Mintz C D: Molecular Mechanisms of Anesthetic Neurotoxicity: A Review of the Current Literature. J Neurosurg Anesthesiol 2016, 28:361-72.
Gentry K R, Steele L M, Sedensky M M, Morgan P G: Early developmental exposure to volatile anesthetics causes behavioral defects in Caenorhabditis elegans. Anesth Analg 2013, 116:185-9.
Jevtovic-Todorovic V, Hartman R E, Izumi Y, Benshoff N D, Dikranian K, Zorumski C F, Olney J W, Wozniak D F: Early exposure to common anesthetic agents causes widespread neurodegeneration in the developing rat brain and persistent learning deficits. J Neurosci 2003, 23:876-82.
Brambrink A M, Evers A S, Avidan M S, Farber N B, Smith D J, Zhang X, Dissen G A, Creeley C E, Olney J W: Isoflurane-induced neuroapoptosis in the neonatal rhesus macaque brain. Anesthesiology 2010, 112:834-41.
Flick R P, Katusic S K, Colligan R C, Wilder R T, Voigt R G, Olson M D, Sprung J, Weaver A L, Schroeder D R, Warner D O: Cognitive and behavioral outcomes after early exposure to anesthesia and surgery. Pediatrics 2011, 128:e1053-61.
Wilder R T, Flick R P, Sprung J, Katusic S K, Barbaresi W J, Mickelson C, Gleich S J, Schroeder D R, Weaver A L, Warner D O: Early exposure to anesthesia and learning disabilities in a population-based birth cohort. Anesthesiology 2009, 110:796-804.
Kuehn B M: FDA considers data on potential risks of anesthesia use in infants, children. JAMA 2011, 305:1749-50, 53.
DiMaggio C, Sun L S, Kakavouli A, Byrne M W, Li G: A retrospective cohort study of the association of anesthesia and hernia repair surgery with behavioral and developmental disorders in young children. J Neurosurg Anesthesiol 2009, 21:286-91.
DiMaggio C, Sun L S, Li G: Early childhood exposure to anesthesia and risk of developmental and behavioral disorders in a sibling birth cohort. Anesth Analg 2011, 113:1143-51.
Ing C, DiMaggio C, Whitehouse A, Hegarty M K, Brady J, von Ungern-Sternberg B S, Davidson A, Wood A J, Li G, Sun L S: Long-term differences in language and cognitive function after childhood exposure to anesthesia. Pediatrics 2012, 130:e476-85.
Hu D, Flick R P, Zaccariello M J, Colligan R C, Katusic S K, Schroeder D R, Hanson A C, Buenvenida S L, Gleich S J, Wilder R T, Sprung J, Warner D O: Association between Exposure of Young Children to Procedures Requiring General Anesthesia and Learning and Behavioral Outcomes in a Population-based Birth Cohort. Anesthesiology 2017, 127:227-40.
Alvarado M C, Murphy K L, Baxter M G: Visual recognition memory is impaired in rhesus monkeys repeatedly exposed to sevoflurane in infancy. Br J Anaesth 2017, 119:517-23.
Raper J, De Biasio J C, Murphy K L, Alvarado M C, Baxter M G: Persistent alteration in behavioural reactivity to a mild social stressor in rhesus monkeys repeatedly exposed to sevoflurane in infancy. Br J Anaesth 2018, 120:761-7.
Ikonomidou C, Bosch F, Miksa M, Bittigau P, Vockler J, Dikranian K, Tenkova T I, Stefovska V, Turski L, Olney J W: Blockade of NMDA receptors and apoptotic neurodegeneration in the developing brain. Science 1999, 283:70-4.
Yon J H, Daniel-Johnson J, Carter L B, Jevtovic-Todorovic V: Anesthesia induces neuronal cell death in the developing rat brain via the intrinsic and extrinsic apoptotic pathways. Neuroscience 2005, 135:815-27.
Ma D, Williamson P, Januszewski A, Nogaro M C, Hossain M, Ong L P, Shu Y, Franks N P, Maze M: Xenon mitigates isoflurane-induced neuronal apoptosis in the developing rodent brain. Anesthesiology 2007, 106:746-53.
Slikker W, Jr., Zou X, Hotchkiss C E, Divine R L, Sadovova N, Twaddle N C, Doerge D R, Scallet A C, Patterson T A, Hanig J P, Paule M G, Wang C: Ketamine-induced neuronal cell death in the perinatal rhesus monkey. Toxicol Sci 2007, 98:145-58.

Kahraman S, Zup S L, McCarthy M M, Fiskum G: GABAergic mechanism of propofol toxicity in immature neurons. J Neurosurg Anesthesiol 2008, 20:233-40.
Fredriksson A, Ponten E, Gordh T, Eriksson P: Neonatal exposure to a combination of N-methyl-Daspartate and gamma-aminobutyric acid type A receptor anesthetic agents potentiates apoptotic neurodegeneration and persistent behavioral deficits. Anesthesiology 2007, 107:427-36.
Franks N P: General anaesthesia: from molecular targets to neuronal pathways of sleep and arousal. Nat Rev Neurosci 2008, 9:370-86.
Solt K, Forman S A: Correlating the clinical actions and molecular mechanisms of general anesthetics. Curr Opin Anaesthesiol 2007, 20:300-6.
Campagna J A, Miller K W, Forman S A: Mechanisms of actions of inhaled anesthetics. N Engl J Med 2003, 348:2110-24.
Rudolph U, Antkowiak B: Molecular and neuronal substrates for general anaesthetics. Nat Rev Neurosci 2004, 5:709-20.
Hemmings H C, Jr., Akabas M H, Goldstein P A, Trudell J R, Orser B A, Harrison N L: Emerging molecular mechanisms of general anesthetic action. Trends Pharmacol Sci 2005, 26:503-10.
Fang M, Tao Y X, He F, Zhang M, Levine C F, Mao P, Tao F, Chou C L, Sadegh-Nasseri S, Johns R A: Synaptic PDZ domain-mediated protein interactions are disrupted by inhalational anesthetics. J Biol Chem 2003, 278:36669-75.
Tao F, Chen Q, Sato Y, Skinner J, Tang P, Johns R A: Inhalational anesthetics disrupt postsynaptic density protein-95, Drosophila disc large tumor suppressor, and zonula occludens-1 domain protein interactions critical to action of several excitatory receptor channels related to anesthesia. Anesthesiology 2015, 122:776-86.
Tao F, Johns R A: Effect of disrupting N-methyl-d-aspartate receptor-postsynaptic density protein-95 interactions on the threshold for halothane anesthesia in mice. Anesthesiology 2008, 108:882-7.
Nikonenko I, Boda B, Steen S, Knott G, Welker E, Muller D: PSD-95 promotes synaptogenesis and multiinnervated spine formation through nitric oxide signaling. J Cell Biol 2008, 183:1115-27.
Kornau H C, Schenker L T, Kennedy M B, Seeburg P H: Domain Interaction between Nmda Receptor Subunits and the Postsynaptic Density Protein Psd-95. Science 1995, 269:1737-40.
Risher W C, Ustunkaya T, Singh Alvarado J, Eroglu C: Rapid Golgi analysis method for efficient and unbiased classification of dendritic spines. PLoS One 2014, 9:e107591.
Fiala J C: Reconstruct: a free editor for serial section microscopy. J Microsc 2005, 218:52-61.
Clark R E, Zola S M, Squire L R: Impaired recognition memory in rats after damage to the hippocampus. J Neurosci 2000, 20:8853-60.
Baker K B, Kim J J: Effects of stress and hippocampal NMDA receptor antagonism on recognition memory in rats. Learn Mem 2002, 9:58-65.
Reger M L, Hovda D A, Giza C C: Ontogeny of Rat Recognition Memory measured by the novel object recognition task. Dev Psychobiol 2009, 51:672-8.
Taylor D L, Joashi U C, Sarraf C, Edwards A D, Mehmet H: Consequential apoptosis in the cerebellum following injury to the developing rat forebrain. Brain Pathol 2006, 16:195-201.
Martone M E, Jones Y Z, Young S J, Ellisman M H, Zivin J A, Hu B R: Modification of postsynaptic densities after transient cerebral ischemia: a quantitative and three-dimensional ultrastructural study. J Neurosci 1999, 19:1988-97.
Gonzalez-Burgos I, Velazquez-Zamora D A, Beas-Zarate C: Damage and plasticity in adult rat hippocampal trisynaptic circuit neurons after neonatal exposure to glutamate excitotoxicity. Int J Dev Neurosci 2009, 27:741-5.
Pigott B M, Garthwaite J: Nitric Oxide Is Required for L-Type Ca(2+) Channel-Dependent Long-Term Potentiation in the Hippocampus. Front Synaptic Neurosci 2016, 8:17.
Benice T S, Rizk A, Kohama S, Pfankuch T, Raber J: Sex-differences in age-related cognitive decline in C57BL/6J mice associated with increased brain microtubule-associated protein 2 and synaptophysin immunoreactivity. Neuroscience 2006, 137:413-23.
Funke L, Dakoji S, Bredt D S: Membrane-associated guanylate kinases regulate adhesion and plasticity at cell junctions. Annu Rev Biochem 2005, 74:219-45.
Sheng M, Hoogenraad C C: The postsynaptic architecture of excitatory synapses: a more quantitative view. Annu Rev Biochem 2007, 76:823-47.
Fiorentini M, Nielsen A K, Kristensen O, Kastrup J S, Gajhede M: Structure of the first PDZ domain of human PSD-93. Acta Crystallogr Sect F Struct Biol Cryst Commun 2009, 65:1254-7.
Carlisle H J, Fink A E, Grant S G, O'Dell T J: Opposing effects of PSD-93 and PSD-95 on long-term potentiation and spike timing-dependent plasticity. J Physiol 2008, 586:5885-900.
Zhu C, Gao J, Karlsson N, Li Q, Zhang Y, Huang Z, Li H, Kuhn H G, Blomgren K: Isoflurane anesthesia induced persistent, progressive memory impairment, caused a loss of neural stem cells, and reduced neurogenesis in young, but not adult, rodents. J Cereb Blood Flow Metab 2010, 30:1017-30.
Kodama M, Satoh Y, Otsubo Y, Araki Y, Yonamine R, Masui K, Kazama T: Neonatal desflurane exposure induces more robust neuroapoptosis than do isoflurane and sevoflurane and impairs working memory. Anesthesiology 2011, 115:979-91.
Briner A, De Roo M, Dayer A, Muller D, Habre W, Vutskits L: Volatile anesthetics rapidly increase dendritic spine density in the rat medial prefrontal cortex during synaptogenesis. Anesthesiology 2010, 112:546-56.
Blanpied T A, Ehlers M D: Microanatomy of dendritic spines: emerging principles of synaptic pathology in psychiatric and neurological disease. Biol Psychiatry 2004, 55:1121-7.
Vickers C A, Stephens B, Bowen J, Arbuthnott G W, Grant S G, Ingham C A: Neurone specific regulation of dendritic spines in vivo by post synaptic density 95 protein (PSD-95). Brain Res 2006, 1090:89-98.
Nikonenko I, Jourdain P, Alberi S, Toni N, Muller D: Activity-induced changes of spine morphology. Hippocampus 2002, 12:585-91.
Briner A, Nikonenko I, De Roo M, Dayer A, Muller D, Vutskits L: Developmental Stage-dependent persistent impact of propofol anesthesia on dendritic spines in the rat medial prefrontal cortex. Anesthesiology 2011, 115:282-93.
Zimering J H, Dong Y, Fang F, Huang L, Zhang Y, Xie Z: Anesthetic Sevoflurane Causes Rho-Dependent Filopodial Shortening in Mouse Neurons. PLoS One 2016, 11:e0159637.
Head B P, Patel H H, Niesman I R, Drummond J C, Roth D M, Patel P M: Inhibition of p75 neurotrophin receptor attenuates isoflurane-mediated neuronal apoptosis in the neonatal central nervous system. Anesthesiology 2009, 110:813-25.
Lemkuil B P, Head B P, Pearn M L, Patel H H, Drummond J C, Patel P M: Isoflurane neurotoxicity is mediated by p75NTR-RhoA activation and actin depolymerization. Anesthesiology 2011, 114:49-57.
Platholi J, Herold K F, Hemmings H C, Jr., Halpain S: Isoflurane reversibly destabilizes hippocampal dendritic spines by an actin-dependent mechanism. PLoS One 2014, 9:e102978.
Amrock L G, Starner M L, Murphy K L, Baxter M G: Long-term effects of single or multiple neonatal sevoflurane exposures on rat hippocampal ultrastructure. Anesthesiology 2015, 122:87-95.
Migaud M, Charlesworth P, Dempster M, Webster L C, Watabe A M, Makhinson M, He Y, Ramsay M F, Morris R G, Morrison J H, O'Dell T J, Grant S G: Enhanced long-term potentiation and impaired learning in mice with mutant postsynaptic density-95 protein. Nature 1998, 396:433-9.
Cooke S F, Bliss T V: Plasticity in the human central nervous system. Brain 2006, 129:1659-73.
Shih J, May L D, Gonzalez H E, Lee E W, Alvi R S, Sall J W, Rau V, Bickler P E, Lalchandani G R, Yusupova M, Woodward E, Kang H, Wilk A J, Carlston C M, Mendoza M V, Guggenheim J N, Schaefer M, Rowe A M, Stratmann G: Delayed environmental enrichment reverses sevoflurane-induced memory impairment in rats. Anesthesiology 2012, 116:586-602.
Stratmann G, Sall J W, Eger E I, 2nd, Laster M J, Bell J S, May L D, Eilers H, Krause M, Heusen F, Gonzalez H E: Increasing the duration of isoflurane anesthesia decreases the minimum alveolar anesthetic concentration in 7-day-old but not in 60-day-old rats. Anesth Analg 2009, 109:801-6.
Stratmann G, May L D, Sall J W, Alvi R S, Bell J S, Ormerod B K, Rau V, Hilton J F, Dai R, Lee M T, Visrodia K H, Ku B, Zusmer E J, Guggenheim J, Firouzian A: Effect of hypercarbia and isoflurane on brain cell death and neurocognitive dysfunction in 7-day-old rats. Anesthesiology 2009, 110:849-61.
Stratmann G, Lee J, Sall J W, Lee B H, Alvi R S, Shih J, Rowe A M, Ramage T M, Chang F L, Alexander T G, Lempert D K, Lin N, Siu K H, Elphick S A, Wong A, Schnair C I, Vu A F, Chan J T, Zai H, Wong M K, Anthony A M, Barbour K C, Ben-Tzur D, Kazarian N E, Lee J Y, Shen J R, Liu E, Behniwal G S, Lammers C R, Quinones Z, Aggarwal A, Cedars E, Yonelinas A P, Ghetti S: Effect of general anesthesia in infancy on long-term recognition memory in humans and rats. Neuropsychopharmacology 2014, 39:2275-87.
Felderhoff-Mueser U, Bittigau P, Sifringer M, Jarosz B, Korobowicz E, Mahler L, Piening T, Moysich A, Grune T, Thor F, Heumann R, Buhrer C, Ikonomidou C: Oxygen causes cell death in the developing brain. Neurobiol Dis 2004, 17:273-82.
Sattler R, Xiong Z, Lu W Y, Hafner M, MacDonald J F, Tymianski M: Specific coupling of NMDA receptor activation to nitric oxide neurotoxicity by PSD-95 protein. Science 1999, 284:1845-8.
Tao F, Su Q, Johns R A: Cell-permeable peptide Tat-PSD-95 PDZ2 inhibits chronic inflammatory pain behaviors in mice. Mol Ther 2008, 16:1776-82.
Aarts M, Liu Y, Liu L, Besshoh S, Arundine M, Gurd J W, Wang Y T, Salter M W, Tymianski M: Treatment of ischemic brain damage by perturbing NMDA receptor-PSD-95 protein interactions. Science 2002, 298:846-50.
Warner D O: Cognitive and behavioral outcomes after early exposure to anesthesia and surgery. Pediatrics 2011, 128:e1053-61.
Ing C, Brambrink A M: Mayo Anesthesia Safety in Kids continued: two new studies and a potential redirection of the field. Br J Anaesth 2019, 122:716-9.
Sprung J, Flick R P, Katusic S K, Colligan R C, Barbaresi W J, Bojanic K, Welch T L, Olson M D, Hanson A C, Schroeder D R, Wilder R T, Warner D O: Attention-deficit/hyperactivity disorder after early exposure to procedures requiring general anesthesia. Mayo Clin Proc 2012, 87:120-9.
Ing C, Hegarty M K, Perkins J W, Whitehouse A J O, DiMaggio C J, Sun M, Andrews H, Li G, Sun L S, von Ungern-Sternberg B S: Duration of general anaesthetic exposure in early childhood and long-term language and cognitive ability. Br J Anaesth 2017, 119:532-40.
Sun L S, Li G, Miller T L, Salorio C, Byrne M W, Bellinger D C, Ing C, Park R, Radcliffe J, Hays S R, DiMaggio C J, Cooper T J, Rauh V, Maxwell L G, Youn A, McGowan F X: Association Between a Single General Anesthesia Exposure Before Age 36 Months and Neurocognitive Outcomes in Later Childhood. JAMA 2016, 315:2312-20.
Warner D O, Zaccariello M J, Katusic S K, Schroeder D R, Hanson A C, Schulte P J, Buenvenida S L, Gleich S J, Wilder R T, Sprung J, Hu D, Voigt R G, Paule M G, Chelonis J J, Flick R P: Neuropsychological and Behavioral Outcomes after Exposure of Young Children to Procedures Requiring General Anesthesia: The Mayo Anesthesia Safety in Kids (MASK) Study. Anesthesiology 2018, 129:89-105.
Warner D O, Chelonis J J, Paule M G, Frank R D, Lee M, Zaccariello M J, Katusic S K, Schroeder D R, Hanson A C, Schulte P J, Wilder R T, Sprung J, Flick R P: Performance on the Operant Test Battery in young children exposed to procedures requiring general anaesthesia: the MASK study. Br J Anaesth 2019, 122:470-9.
Davidson A J, Disma N, de Graaff J C, Withington D E, Dorris L, Bell G, Stargatt R, Bellinger D C, Schuster T, Arnup S J, Hardy P, Hunt R W, Takagi M J, Giribaldi G, Hartmann P L, Salvo I, Morton N S, von Ungern Sternberg B S, Locatelli B G, Wilton N, Lynn A, Thomas J J, Polaner D, Bagshaw O, Szmuk P, Absalom A R, Frawley G, Berde C, Ormond G D, Marmor J, McCann M E, consortium GAS: Neurodevelopmental outcome at 2 years of age after general anaesthesia and awake-regional anaesthesia in infancy (GAS): an international multicentre, randomised controlled trial. Lancet 2016, 387:239-50.
McCann M E, de Graaff J C, Dorris L, Disma N, Withington D, Bell G, Grobler A, Stargatt R, Hunt R W, Sheppard S J, Marmor J, Giribaldi G, Bellinger D C, Hartmann P L, Hardy P, Frawley G, Izzo F, von Ungern Sternberg B S, Lynn A, Wilton N, Mueller M, Polaner D M, Absalom A R, Szmuk P, Morton N, Berde C, Soriano S, Davidson A J, Consortium GAS: Neurodevelopmental outcome at 5 years of age after general anaesthesia or awake-regional anaesthesia in infancy (GAS): an international, multicentre, randomised, controlled equivalence trial. Lancet 2019, 393:664-77.
Jevtovic-Todorovic V: Exposure of Developing Brain to General Anesthesia: What Is the Animal Evidence? Anesthesiology 2018, 128:832-9.
Disma N, O'Leary J D, Loepke A W, Brambrink A M, Becke K, Clausen N G, De Graaff J C, Liu F, Hansen T G, McCann M E, Salorio C F, Soriano S, Sun L S, Szmuk P, Warner D O, Vutskits L, Davidson A J: Anesthesia and the developing brain: A way forward for laboratory and clinical research. Paediatr Anaesth 2018, 28:758-63.
Lin E P, Lee J R, Lee C S, Deng M, Loepke A W: Do anesthetics harm the developing human brain? An integrative analysis of animal and human studies. Neurotoxicol Teratol 2017, 60:117-28.
Schaefer M L, Wang M, Perez P J, Coca Peralta W, Xu J, Johns R A: Nitric Oxide Donor Prevents Neonatal Isoflurane-induced Impairments in Synaptic Plasticity and Memory. Anesthesiology 2019, 130:247-62.
Ennaceur A, Delacour J: A new one-trial test for neurobiological studies of memory in rats. 1: Behavioral data. Behav Brain Res 1988, 31:47-59.
Kang E, Jiang D, Ryu Y K, Lim S, Kwak M, Gray C D, Xu M, Choi J H, Junn S, Kim J, Xu J, Schaefer M, Johns R A, Song H, Ming G L, Mintz C D: Early postnatal exposure to isoflurane causes cognitive deficits and disrupts development of newborn hippocampal neurons via activation of the mTOR pathway. PLoS Biol 2017, 15:e2001246.
Shirai Y, Kouzuki T, Kakefuda K, Moriguchi S, Oyagi A, Hone K, Morita S Y, Shimazawa M, Fukunaga K, Takeda J, Saito N, Hara H: Essential role of neuron-enriched diacylglycerol kinase (DGK), DGKbeta in neurite spine formation, contributing to cognitive function. PLoS One 2010, 5:e11602.
Daumas S, Halley H, Frances B, Lassalle J M: Encoding, consolidation, and retrieval of contextual memory: differential involvement of dorsal CA3 and CA1 hippocampal subregions. Learn Mem 2005, 12:375-82.
Phillips R G, LeDoux J E: Differential contribution of amygdala and hippocampus to cued and contextual fear conditioning. Behav Neurosci 1992, 106:274-85.
Rabbitts J A, Groenewald C B, Moriarty J P, Flick R: Epidemiology of ambulatory anesthesia for children in the United States: 2006 and 1996. Anesth Analg 2010, 111:1011-5.
Tzong K Y, Han S, Roh A, Ing C: Epidemiology of pediatric surgical admissions in U S children: data from the HCUP kids inpatient database. J Neurosurg Anesthesiol 2012, 24:391-5.
Zaccariello M J, Frank R D, Lee M, Kirsch A C, Schroeder D R, Hanson A C, Schulte P J, Wilder R T, Sprung J, Katusic S K, Flick R P, Warner D O: Patterns of neuropsychological changes after general anaesthesia in young children: secondary analysis of the Mayo Anesthesia Safety in Kids study. Br J Anaesth 2019, 122:671-81.
Shao L J, Zou Y, Xue F S: Anesthesia-induced developmental neurotoxicity in children: past, present, and future. Chin Med J (Engl) 2019, 132:1990-2.
Communication FDS: Drug safety communication: FDA approves label changes for use of general anesthetic and sedation drugs in young children. Apr. 27, 2017.
Communication FDS: FDA review results in new warnings about using general anesthetics and sedation drugs in young children and pregnant women. Dec. 14, 2016.
Grienberger C, Chen X, Konnerth A: Dendritic function in vivo. Trends Neurosci 2015, 38:45-54.
Bourne J, Harris K M: Do thin spines learn to be mushroom spines that remember? Curr Opin Neurobiol 2007, 17:381-6.
Hafting T, Fyhn M, Molden S, Moser M B, Moser E I: Microstructure of a spatial map in the entorhinal cortex. Nature 2005, 436:801-6.
Kraeuter A K, Guest P C, Sarnyai Z: The Y-Maze for Assessment of Spatial Working and Reference Memory in Mice. Methods Mol Biol 2019, 1916:105-11.
Satomoto M, Satoh Y, Terui K, Miyao H, Takishima K, Ito M, Imaki J: Neonatal exposure to sevoflurane induces abnormal social behaviors and deficits in fear conditioning in mice. Anesthesiology 2009, 110:628-37.
Man Y G, Zhou R G, Zhao B: Efficacy of rutin in inhibiting neuronal apoptosis and cognitive disturbances in sevoflurane or propofol exposed neonatal mice. Int J Clin Exp Med 2015, 8:14397-409.
Gonzales E L, Yang S M, Choi C S, Mabunga D F, Kim H J, Cheong J H, Ryu J H, Koo B N, Shin C Y: Repeated neonatal propofol administration induces sex-dependent long-term impairments on spatial and recognition memory in rats. Biomol Ther (Seoul) 2015, 23:251-60.
Boscolo A, Ori C, Bennett J, Wiltgen B, Jevtovic-Todorovic V: Mitochondrial protectant pramipexole prevents sex-specific long-term cognitive impairment from early anaesthesia exposure in rats. Br J Anaesth 2013, 110 Suppl 1:i47-52.
Kim J J, Fanselow M S: Modality-specific retrograde amnesia of fear. Science 1992, 256:6757.

Although the foregoing subject matter has been described in some detail by way of illustration and example for purposes of clarity of understanding, it will be understood by those skilled in the art that certain changes and modifications can be practiced within the scope of the appended claims.

1. A method for treating or preventing a cognitive impairment in a subject, the method comprising administering to the subject an agent that enhances the NO-cGMP-PKG pathway.

2. The method of claim 1, wherein the agent that enhances the NO-cGMP-PKG pathway includes an NO donor, a guanylate cyclase activator, and a type 5 phosphodiesterase (PDE5) inhibitor.

3. The method of claim 1, comprising administering to the subject a therapeutically effective amount of at least one nitric oxide (NO) donor.

4. The method of claim 1, wherein the cognitive impairment is associated with one or more surgical procedures.

5. The method of claim 1, wherein the cognitive impairment is anesthetic induced.

6. The method of claim 5, wherein the anesthetic is a general anesthetic or a regional anesthetic.

7. The method of claim 6, wherein the general anesthetic is selected from the group consisting of an inhalational anesthetic, an injectable anesthetic, and combinations thereof.

8. The method of claim 7, wherein the inhalational anesthetic is selected from the group consisting of isoflurane ((RS)-2-chloro-2-(difluoromethoxy)-1,1,1-trifluoroethane), halothane (2-bromo-2-chloro-1,1,1-trifluoroethane), sevoflurane (1,1,1,3,3,3-hexafluoro-2-(fluoromethoxy)propane), desflurane (1,2,2,2-tetrafluoroethyl difluoromethyl ether), enflurane (2-chloro-1,1,2,-trifluoroethyl-difluoromethyl ether), methoxyflurane (2,2-dichloro-1,1-difluoroethyl methyl ether), nitrous oxide, xenon, and combinations thereof.

9. The method of claim 7, wherein the injectable anesthetic is selected from the group consisting of propofol (2,6-diisopropylphenol), etomidate (ethyl 3-[(1R)-1-phenylethyl]imidazole-5-carboxylate), ketamine ((RS)-2-(2-Chlorophenyl)-2-(methylamino)cyclohexanone), a barbiturate, a benzodiazepine, and combinations thereof.

10. The method of claim 6, wherein the regional anesthetic is selected from the group consisting of a nerve block, a spinal anesthetic, an epidural anesthetic, and a caudal anesthetic.

11. The method of claim 1, wherein the cognitive impairment is selected from the group consisting of an impaired memory, an impaired object recognition memory, a learning disability, and an attention deficit/hyperactivity disorder.

12. The method of claim 1, further comprising one or more conditions or disorders selected from the group consisting of anxiety and emotional reactivity.

13. The method of claim 1, wherein the cognitive impairment is associated with a change or impairment in dendritic spine morphology or development; synaptic plasticity; neural plasticity; long-term potentiation (LTP), neuronal apoptosis, and combinations thereof.

14. The method of claim 1, wherein the cognitive impairment is associated with a disruption of PSD-95 PDZ2 domain-mediated protein-protein interactions and/or a N-methyl-D aspartate (NMDA) receptor/PSD-95 PDZ2/neuronal nitric oxide synthase (nNOS) signaling pathway.

15. The method of claim 3, wherein the at least one NO donor is selected from the group consisting of sodium nitroprusside (SNP), nitroglycerin (NTG), an organic nitrate, a sydnonimine, a diazeniumdiolate, an S-nitrosothiol, and nitric oxide (NO).

16. The method of claim 15, wherein the at least one NO donor is a sydnonimine.

17. The method of claim 16, wherein the sydnonimine is molsidomine or isosorbide.

18. The method of claim 17, further comprising a pharmaceutical formulation comprising molsidomine, isosorbide, or other NO donor.

19. The method of claim 2, wherein the guanylate cyclase activator is selected from the group consisting of 3-[2-[(4-Chlorophenyl)thiophenyl]-N-[4-(dimethylamino)butyl]-2-propenamide hydrochloride (A-350619 hydrochloride); 5-Cyclopropyl-2-[1-[(2-fluorophenyl)methyl]-1H-pyrazolo[3,4-b]pyridin-3-yl]-4-pyrimidinamine (BAY-41-2272); 2-[1-[(2-Fluorophenyl)methyl]-1H-pyrazolo[3,4-b]pyridin-3-yl]-5-(4-morpholinyl)-4,6-pyrimidinediamine (BAY-41-8543); 4-[[(4-Carboxybutyl)[2-[2-[[4-(2-phenylethyl)phenyl]methoxy]phenyl]ethyl]amino]methyl]benzoic acid hydrochloride (Cinaciguat hydrochloride; BAY-58-2667 hydrochloride); Guanylin; Amino-3-morpholinyl-1,2,3-oxadiazolium chloride (SIN-1 chloride); 3-(5′-Hydroxymethyl-2′-furyl)-1-benzyl indazole (YC-1); 3-Bromo-4-methyl-3,4-hexamethylene-3,4-dihydrodiazete 1,2-dioxide (DD2); and 8,13-Divinyl-3,7,12,17-tetramethyl-21H,23H-porphine-2,18-dipropionic acid (Protoporphyrin IX).

20. The method of claim 2, wherein the PDE5 inhibitor is selected from the group consisting of sildenafil, tadalafil, vardenafil, avanafil, mirodenafil, udenafil, lodenafil, (3-Chlorophenylamino)-4-phenylphthalazine (MY-5445), 1,2-Dihydro-2-[(2-methyl-4-pyridinyl)methyl]-1-oxo-8-(2-pyrimdinylmethoxy)-4-(3,4,5-trimethoxyphenyl)-2,7-naphthyridine-3-carboxylic acid methyl ester hydrochloride (T 0156 hydrochloride), 5-[2-Ethoxy-5-[(4-ethyl-1-piperazinyl)sulfonyl]-3-pyridinyl]-3-ethyl-2,6-dihydro-2-(2-methoxyethyl)-7H-pyrazolo[4,3-d]pyrimidin-7-one benzenesulfonate (gisadenafil besylate), 2,6-bis(Diethanolamino)-4,8-dipiperidinopyrimido[5,4-d]pyrimidine (dipyridamole), 2-(2-Propyloxyphenyl)-8-azapurin-6-one (zaprinast), and cGMP Dependent Kinase Inhibitor Peptide.

21. The method of claim 1, wherein the agent that enhances the NO-cGMP-PKG pathway is administered in combination with one or more anesthetics.

22. The method of claim 21, wherein the agent that enhances the NO-cGMP-PKG pathway is administered before, after, or concurrently with one or more anesthetics.

23. The method of claim 1, wherein the subject is selected from the group consisting of a neonate, an infant, a one- to three-year old child, an unborn fetus, and a patient who is pregnant.
